SEQUENCE LISTING (18 sequences)

SEQ ID NO: 1 - protein, 1449 aa, synthetic construct
  REGION 1..1449: note = GTF 0768 mature protein with two N-terminal residues
VADQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 2 - protein, 1447 aa, Leuconostoc pseudomesenteroides
  REGION 1..1447: note = GTF 0768 mature wildtype protein
DQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 3 - protein, 1341 aa, synthetic construct
  REGION 1..1341: note = vGTFJ protein
VIDGKYYNANEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSYNDNHYNDKTDGAALAMENKQRLALLFSLAKPIKERTPAVSPLYNNTFNTTQRDEKTDWINKDGSKAYNEDGTVKLSTIGKYNEKYGDASGNYVYIRAHDNNVQDIIAEIIKAEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYSTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFQTGDNNWYYIGANGKSVTGFVKIGDDTYFFAKDGKQVKGQTVSAGNGRISYYYGDSGKRAVSTWIEIQPGVYVYFDKNGIAYPPRVLN

SEQ ID NO: 4 - protein, 1341 aa, synthetic construct
  REGION 1..1341: note = Variant of GTF 6855 protein
VIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSYNDNHYNDKTDGAALAMENKQRLALLFSLAKPIKERTPAVSPLYNNTFNTTQRDEKTDWINKDGSKAYNEDGTVKQSTIGKYNEKYGDASGNYVYVRAHDNNVQDIIAEIIKAEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYSTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDEYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQHLYFNADGSQVKGGVVKNADGTYSKYKEGSGELVTNEFFTTDGNVWYYAGANGKTVTGAQVINGQHLYFNADGSQVKGGVVKNADGTYSKYKEGSGELVTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 5 - protein, 1341 aa, Streptococcus salivarius [sequence omitted]

SEQ ID NO: 6 - protein, 1341 aa, Streptococcus salivarius
  REGION 1..1341: note = GTF 7527. Position-1 methionine added.
MIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLNLDQSAKLNVEMGKIHANQKYRALIVGTADGIKNFTSDADAIAAGYVKETDSNGVLTFGANDIKGYETFDMSGFVAVWVPVGASDNQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGKDYQAKYGGEFLAELKAKYPEMFKVNMISTGKPIDDSVKLKQWKAEYFNGTNVLERGVGYVLSDEATGKYFTVTKEGNFIPLQLTGKEKVITGFSSDGKGITYFGTSGTQAKSAFVTFNGNTYYFDARGHMVTNSEYSPNGKDVYRFLPNGIMLSNAFYIDANGNTYLYNSKGQMYKGGYTKFDVSETDKDGKESKVVKFRYFTNEGVMAKGVTVIDGFTQYFGEDGFQAKDKLVTFKGKTYYFDAHTGNGIKDTWRNINGKWYYFDANGVAATGAQVINGQKLYFNEDGSQVKGGVVKNADGTYSKYKEGFGELVTNEFFTTDGNVWYYAGANGKTVTGAQVINGQHLYFNADGSQVKGGVVKNADGTYSKYNASTGERLTNEFFTTGDNNWYYIGANGKSVTGEVKIGDDTYFFAKDGKQVKGQTVSAGNGRISYYYGDSGKRAVSTWIEIQPGVYVYFDKNGLAYPPRVLN

SEQ ID NO: 7 - protein, 1341 aa, Streptococcus salivarius
  REGION 1..1341: note = GTF 2678 protein. Position-1 methionine added.
MTDGKYYYVNEDGSHKENFAITVNGQLLYFGKDGALTSSSTHSFTPGTTNIVDGFSINNRAYDSSEASFELINGYLTADSWYRPVSIIKDGVTWQASTAEDFRPLLMAWWPNVDTQVNYLNYMSKVFNLEAKYTSTDKQADLNRAAKDIQVKIEQKIQAEKSTQWLRETISAFVKTQPQWNKETENYSKGGGEDHLQGGALLYVNDSRTPWANSNYRLLNRTATNQTGTINKSVLDEQSDPNHMGGFDFLLANDVDLSNPVVQAEQLNQIHYLMNWGSIVMGDKDANFDGIRVDAVDNVNADMLQLYTNYFREYYGVNKSEAQALAHISVLEAWSLNDNHYNDKTDGAALAMENKQRLALLFSLAKPIKDRTPAVSPLYNNTFNTTQRDFKTDWINKDGSTAYNEDGTAKQSTIGKYNEKYGDASGNYVFIRAHDNNVQDIIAEIIKKEINKKSDGFTISDSEMKQAFEIYNKDMLSSNKKYTLNNIPAAYAVMLQNMETITRVYYGDLYTDDGHYMETKSPYHDTIVNLMKNRIKYVSGGQAQRSYWLPTDGKMDNSDVELYRTSEVYTSVRYGKDIMTADDTEGSKYSRTSGQVTLVVNNPKLTLHESAKLNVEMGKIHANQKYRALIVGTADGIKNFTSDAEAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANSKSSGRDYQAQYGGEFLAELKAKYPKMFTENMISTGKPIDDSVKLKQWKAKYFNGTNVLDRGVGYVLSDEATGKYFTVTKEGNFIPLQLTGNEKAVTGFSNDGKGITYFGTSGNQAKSAFVTFNGNTYYFDARGHMVTNGEYSPNGKDVYRFLPNGIMLSNAFYVDANGNTYLYNYKGQMYKGGYTKFDVTETDKDGNESKVVKFRYFTNEGVMAKGLTVIDGSTQYFGEDGFQTKDKLATYKGKTYYFEAHTGNAIKNTWRNIDGKWYHFDENGVAATGAQVINGQKLYFNEDGSQVKGGVVKNADGTYSKYKEGSGELVTNEFFTTDGNVWYYAGADGKTVTGAQVINGQHLYFKEDGSQVKGGVVKNADGTYSKYDAATGERLTNEFFTTGDNNWYYIGSNGKTVTGEVKIGADTYYFAKDGKQVKGQTVTAGNGRISYYYGDSGKKAISTWIEIQPGIYVYFDKTGIAYPPRVLN

SEQ ID NO: 8 - protein, 1340 aa, Streptococcus salivarius
  REGION 1..1340: note = GTF 2919 protein. Position-1 methionine added.
MIDGKYYYVNKDGSHKENFAITVNGQLLYFGKDGALTSSSTYSFTQGTTNIVDGFSKNNRAYDSSEASFELIDGYLTADSWYRPVSIIKDGVTWQASTKEDFRPLLMAWWPNVDTQVNYLNYMSKVFNLDAKYTSTDKQVDLNRAAKDIQVKIEQKIQAEKSTQWLREAISAFVKTQPQWNKETENFSKGGGEDHLQGGALLYVNDPRTPWANSNYRLLNRTATNQTGTIDKSVLDEQSDPNHMGGFDFLLANDVDTSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKKAFEIYNKDMLSSDKKYTLNNIPAAYAVMLQNMETITRVYYGDLYTDDGHYMETKSPYYDTIVNLMKNRIKYVSGGQAQRSYWLPTDGKMDKSDVELYRTNEVYTSVRYGKDIMTADDTQGSKYSRTSGQVTLVVNNPKLSLDKSAKLDVEMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNEKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGGVVKNADGTFSKYKDGSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 9 - protein, 1340 aa, unidentified
  REGION 1..1340: note = GTF 2765 protein
  REGION 1..1340: note = GTF 2765 protein. Position-1 methionine added.
MIDGKYYYVNEDGSHKENFAITVNGQLLYFGKDGALTSSSTYSFTQGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSNYRLLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDVAALAMENKQRLALLFSLAKPIKERTPAVSPLYNNTFNTTQRDEKTDWINKDGSKAYNEDGTVKKSTIGKYNEKYGDASGNYVFIRAHDNNVQDIIAEIIKKEINEKSDGFTITDSEMKRAFEIYNKDMLSNDKKYTLNNIPAAYAVMLQNMETITRVYYGDLYTDDGNYMEAKSPYYDTIVNLMKSRIKYVSGGQAQRSYWLPTDGKMDKSDVELYRTNEVYTSVRYGKDIMTADDTQGSKYSRTSGQVTLVVNNPKLTLDQSAKLNVVMGKIHANQKYRALIVGTPNGIKNFTSDAEAIAAGYVKETDGNGVLTFGANDIKGYETFDMSGFVAVWVPVGASDDQDIRVAASTAAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRNALKALHKAGIQAIADWVPDQIYQLPGKEVVTATRTDGAGRKISDAIIDHSLYVANSKSSGKDYQAKYGGEFLAELKAKYPEMFKVNMISTGKPIDDSVKLKQWKAEYFNGTNVLDRGVGYVLSDEATGKYFTVTKEGNFIPLQLKGNKKVITGFSSDGKGITYFGTSGNQAKSAFVTFNGNTYYFDARGHMVTNGEYSPNGKDVYRFLPNGIMLSNAFYVDGNGNTYLYNSKGQMYKGGYSKFDVTETKDGKESKVVKFRYFTNEGVMAKGVTVVDGFTQYFNEDGIQSKDELVTYNGKTYYFEAHTGNAIKNTWRNIKGKWYHFDANGVAATGAQVINGQHLYFNEDGSQVKGSIVKNADGTFSKYKDSSGDLVVNEFFTTGDNVWYYAGANGKTVTGAQVINGQHLFFKEDGSQVKGDFVKNSDGTYSKYDAASGERLTNEFFTTGDNHWYYIGANGKTVTGEVKIGDDTYFFAKDGKQLKGQIVTTRSGRISYYFGDSGKKAISTWVEIQPGVFVFFDKNGLAYPPENMN

SEQ ID NO: 10 - protein, 1518 aa, Streptococcus salivarius
  REGION 1..1518: note = Wild type GTF corresponding to GTF 6855
MENKIHYKLHKVKKQWVTIAVASVALATVLGGLSVTTSSVSADETQDKTVTQSNSGTTASLVTSPEATKEADKRTNTKEADVLTPAKETNAVETATTTNTQATAEAATTATTADVAVAAVPNKEAVVTTDAPAVTTEKAEEQPATVKAEVVNTEVKAPEAALKDSEVEAALSLKNIKNIDGKYYYVNEDGSHKENFAITVNGQLLYFGKDGALTSSSTYSFTPGTTNIVDGFSINNRAYDSSEASFELIDGYLTADSWYRPASIIKDGVTWQASTAEDFRPLLMAWWPNVDTQVNYLNYMSKVFNLDAKYSSTDKQETLKVAAKDIQIKIEQKIQAEKSTQWLRETISAFVKTQPQWNKETENYSKGGGEDHLQGGALLYVNDSRTPWANSDYRRLNRTATNQTGTIDKSILDEQSDPNHMGGFDFLLANDVDLSNPVVQAEQLNQIHYLMNWGSIVMGDKDANFDGIRVDAVDNVDADMLQLYTNYFREYYGVNKSEANALAHISVLEAWSLNDNHYNDKTDGAALAMENKQRLALLFSLAKPIKERTPAVSPLYNNTFNTTQRDEKTDWINKDGSKAYNEDGTVKQSTIGKYNEKYGDASGNYVFIRAHDNNVQDIIAEIIKKEINPKSDGFTITDAEMKQAFEIYNKDMLSSDKKYTLNNIPAAYAVMLQNMETITRVYYGDLYTDDGHYMETKSPYYDTIVNLMKSRIKYVSGGQAQRSYWLPTDGKMDNSDVELYRTNEVYTSVRYGKDIMTANDTEGSKYSRTSGQVTLVANNPKLTLDQSAKLNVEMGKIHANQKYRALIVGTADGIKNFTSDADAIAAGYVKETDSNGVLTFGANDIKGYETFDMSGFVAVWVPVGASDDQDIRVAPSTEAKKEGELTLKATEAYDSQLIYEGFSNFQTIPDGSDPSVYTNRKIAENVDLFKSWGVTSFEMAPQFVSADDGTFLDSVIQNGYAFADRYDLAMSKNNKYGSKEDLRDALKALHKAGIQAIADWVPDQIYQLPGKEVVTATRTDGAGRKIADAIIDHSLYVANTKSSGKDYQAKYGGEFLAELKAKYPEMFKVNMISTGKPIDDSVKLKQWKAEYFNGTNVLERGVGYVLSDEATGKYFTVTKDGNFIPLQLTGNEKVVTGFSNDGKGITYFGTSGTQAKSAFVTFNGNTYYFDARGHMVTNGEYSPNGKDVYRFLPNGIMLSNAFYVDANGNTYLYNSKGQMYKGGYTKFDVTETDKDGKESKVVKFRYFTNEGVMAKGVTVIDGFTQYFGEDGFQAKDKLVTFKGKTYYFDAHTGNAIKDTWRNINGKWYHFDANGVAATGAQVINGQKLYFNEDGSQVKGGVVKNADGTYSKYKEGSGELVTNEFFTTDGNVWYYAGANGKTVTGAQVINGQHLYFNADGSQVKGGVVKNADGTYSKYDASTGERLTNEFFTTGDNNWYYIGANGKSVTGEVKIGDDTYFFAKDGKQVKGQTVSAGNGRISYYYGDSGKRAVSTWIEIQPGVYVYFDKNGIAYPPRVLN

SEQ ID NO: 11 - protein, 1423 aa, Leuconostoc pseudomesenteroides
  source 1..1423: note = 0768 gtf truncated wildtype protein A
DASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 12 - protein, 1375 aa, Leuconostoc pseudomesenteroides
  source 1..1375: note = 0768 gtf truncated wildtype protein B
STEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 13 - protein, 1392 aa, Streptococcus salivarius
INGKQYYVNSDGSVRKNFVFEQDGKSYYFDAETGALATKSQDEFSTEPIKATVDFSSGNQLYKNDDKSLDQLDTFITADAWYRPKSILKDGKTWTASTEADKRPLLMVWWPDKSTQVNYLNYMQNQGLGAGSFSTNSSQESLDQAAKAVQTKIEERIAREGNTNWLRTSIDQFIKTQPGWNSSTENSSYDHLQGGQLLFNNSKGDTGNRTSHANSDYRLLNRTPTNQTGTRKYFKDNSIGGLEFLLANDIDNSNPAVQAEQLNWLHFMMNIGSIMANDPTANFDGLRVDALDNVDADLLQIASDYFKAAYGVDKSEANAIKHLSYLEAWSANDPYYNKDTKGAQLPIDNALRNALTNLLMRDKNTRMQLGDMTAFINSSLNPRGANDKNGERMANYIFTRAHDTEAQTIIQRIIRDRINPNLFGYNFTRDEIKKAFEIYNEDIDKAHKTYASYNLPSVYALMLTNKDSVTRVYYGDLYREDGHYMAKKTPYFDAIDTLLRARIKYVAGGQDMEVKKVGNDGLLTSVRYGKGANNRTDLGTSETRTQGMGVIMTNNYDFRLGSNETVTMNMGRAHRNQLYRPLLLTTKDGIATYLNDSDVPKKLLKRTDWNGNLTFNANDVFGVENVQVSGYLGVWVPYGAKANQDARTQPSNRANSDGQVYKSSAALDSQVMYEAFSNFQAFADDQPELYMNRVLAKNTDLLKAWGITSVGLPPQYVSSKDGTFLDSTIDNGYAFDDRYDMALSQNNKYGSLEDLLNVLRALHKDGIQAIADWVPDQIYNLPGKEVVNATRVNGYGNHQVGYQIIDQAYVANTRTDGTDYQGKYGGAFLDELKAKYPSIFNRVQISNGKQLPTNEKITKWSAKYFNGTNILGRGINYVLRDDKTNQYFNTSANSQLLPTPLRDTGAITSTQVFQRRGQDVYFLRDNQAVKNEFVQDGNGNWYYFGGDGKMTKGAQNINNKDYYFFDNGIQLRNALRRASNGYTYYYGLDGAMVKNAFVDFDDKHKQVRAFTTQGTMVVGNLHWSGHHFYFDRETGIQAKDRVVRTDDGKLHYYVAETGDMGRNVFATDSSTGKRYYFDADGNTVTGSRVIDGKTYYFNQDGSVGTAYSNRADSIIFENGKARYITPAGEIGRSIFVYNPETKAWNYFDKEGNRVTGRQYIDGHLYYFKEDGSQAKGEIIEENGVKYYYEPESGILASGRYLQVGDDQWMYFKQDGSLAIGQVRADHGYLKYFDKNGIQVKGKTVVEDGKTYYYDAHSGALVTSSFAEIAPNQWSYFNTDGQALKGKWTINGKEYYFDENGIQYKGKAIKVGSRYKYYDENDGQPVTNRFAQIEPNVWAYFGADGYAVTGEQVINGQHLYFDQSGRQVKGAYVTVNGQRRYYDANTGEYVPGR

SEQ ID NO: 14 - protein, 1449 aa, synthetic construct
MADQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 15 - protein, 1448 aa, synthetic construct
ADQNVNDPSVATTTQNVVTDQDTSIDASVATTVNPNLDDTQADNTNIQTPTDQNDESKDTTPKVETGDTTNSQSTEAQETTAQTNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 16 - protein, 1365 aa, Leuconostoc pseudomesenteroides
TNNDVETPQNSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 17 - protein, 1356 aa, Leuconostoc pseudomesenteroides
NSDAAIETGLLTTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

SEQ ID NO: 18 - protein, 1345 aa, Leuconostoc pseudomesenteroides
TTNNQIRYVNPDGTVLTGAYKTINGNTYYFDDDSGVALVGLHKIGDTLKGFSLNGVQVKGDYLTAANGDKYYFDSNGNAVSGVQQINGKTYYFDSTGKLMKGYTAVLNGVVTFFNSTTGEADNTDASTIKTGVTIDNSDYTVHNAAYDNTAASFDNINGYLTAESWYRPKEILENGESWRPSTAEDKRPILITWQPDIVTEVNYLNMMAANGLLSINAPFTTASDLAIMNDAVRAVQKNIEMRISQEKSTDWLKALMTQFINTQPQWNEVSESPSNDHLQGGALTYVNSPLTPDANSNFRLLNRTPTNQSGTTRYDTDKSKGGFELLLANDVDNSNPVVQAEQLNWLYYLMNFGSITANDPTANFDGIRVDAVDNVDADLLQIASDYFKLAYGTSLSDTNANQHLSILEDWSANDAEYMSKTGSNQLTMDTYTQQQLLFSLTKQVGNRADMRRFLEYFMINRANDSTENVATPNYSFVRAHDSEVQTVIATIIKDLHPDVVNSLAPTQAQLEEAFAVYNADMNRVDKQYTQYNMPSAYAMLLTNKDTIPRVYYGDLYTDDGEYMGTQTPYYDAIVNLLQSRVKYVAGGQSMAVDQHDILTSVRYGKNLADANATSDDLTSINSGIGVIVSNNPNLSLASGETVVLHMGIAHANQVYREILETTDNGIANNTDIFKTTDSNGDLIFTASEIHGYSNVQVSGFLSVWAPKDATDDQDVRTAASESTSNDGNTLHSNAALDSNLIYEGFSNFQSTPQSESEFANVKIAANVNLFKSWGVTSFQMAPQYRSSTDTSFLDSIIQNGYAFTDRYDLGFETPTKYGTDQQLRDAIKALHANGIQAMADFVPDQIYNLPQTELVSVSRTDSLGNQSANSNAANVLYVSHTVGGGEYQSKYGGEFLAIIKSKYPSLFKTIQVSTGLPIDDSTKIKEWSAKYFNGSNIQGRGFGYVLSDGGTQNYFKVISNSTDDDFLPNQLTGKPTMTGFEQTSKGIVYYSKSGIQAKNQFVKDDVSGNYYYFNKNGLMTVGSKTINGKNYMFLPNGVELRGSFLQTADGTVNYYATNGAQVQDSYVTDTEGNSYYFDGDGEMVTGTYTVDGHAQYFDVNGVQTKGAIITLGGVQRYYQAGNGNLATNQYVSYNNSWYYANTKGELVTGVQSINGNVQYFASNGQQIKGQIVVTGNQKSYYDANTGNLIKNDFLTPDQGKTWYYADQDGNLVVGAQEVNGHKLYFDDNGIQIKDQIISNDGQQYYYQGGNGDLVTNRYISYNDSWYYADATGVLVTGQQIINGETQYFRTDGRQVKGQIIADGDKQHYYDADSGNLVKNNFVTVDQGKTWYYADQDGNLSLVDR

(19) United States
(12) Patent Application Publication
(10) Pub. No.: US 2026/0198541 A1
(43) Pub. Date: Jul. 16, 2026

(54) METHOD FOR IMPROVING FLAVOR IN PLANT-BASED FOOD STUFF

(71) Applicants: INTERNATIONAL N&H DENMARK APS, Kongens Lyngby (DK); DANISCO US INC., Palo Alto, CA (US)

(72) Inventors: Karina Hansen Kjær, Kongens Lyngby (DK); Jacob Franz Ewert, Kongens Lyngby (DK); Slavko Kralj, OEGSTGEEST (NL); Michael Thorsen, Kongens Lyngby (DK); Bettina Høj Hornshøj, Kongens Lyngby (DK); Katja Søholm Hildebrandt, Kongens Lyngby (DK)

(21) Appl. No.: 19/119,910

(22) PCT Filed: Oct. 17, 2023
(86) PCT No.: PCT/US2023/077065

§ 371 Date: Apr. 10, 2025

Related U.S. Application Data
(60) Provisional application No. 63/379,883, filed on Oct. 17, 2022.

(51) Int. Cl. A23L 27/00; A23C 11/10; A23L 29/00; C12P 19/18; C12P 19/60

(52) U.S. Cl. CPC A23L 27/84; A23C 11/106; A23L 27/86; A23L 29/06; C12P 19/18; C12P 19/60

(57) ABSTRACT

Disclosed herein are methods of glucosylating an isoflavone glycoside, as well as methods of reducing off-flavor in a food product/precursor. Further disclosed are compositions comprising one or more glucosylated isoflavone glycosides, such as a food product/precursor

This application claims the benefit of U.S. Provisional Appl. No. 63/379,883 (filed Oct. 17, 2022), which is incorporated herein by reference in its entirety.

### FIELD

The present disclosure is in the field of glycosylation of components present in plant-based food products to improve flavor. For example, the disclosure pertains to glucosylation of isoflavone glycosides in situ in food and food precursors.

### REFERENCE TO SEQUENCE LISTING SUBMITTED ELECTRONICALLY

The official copy of the sequence listing is submitted electronically via EFS-Web as a file named NB42164WOPCT_SequenceListing.xml created on Oct. 16, 2023 and having a size of about 46 kilobytes, and is filed concurrently with the specification. The sequence listing contained in this file is part of the specification and is incorporated herein by reference in its entirety.

### BACKGROUND

Plant-based or non-dairy protein food alternatives, such as soy-, almond-, pea-, bean-, rice- or oat-based products (e.g. milk or fresh fermented products), are one of the fastest growing segments in all food product categories worldwide (Mäkinen et al., 2016, Crit. Rev. Food Sci. Nutr. 56:339-349; Sethi et al., 2016, J. Food Sci. Technol. 53:3408-3423). Off flavors, however, remain a major hurdle in the use of plant-based materials in mainstream food applications. For example, soy-derived materials contain several polyphenolic compounds, such as isoflavones, saponins and phenolic acids that impart undesirable sensory properties such as bitterness and astringency tastes. Today, very cumbersome processes, such as dispersion of material in alkaline conditions followed by membrane separation or size-exclusion chromatography, are tried and used to remove these components and hence their unwanted flavor (Damodaran et al., 2013, Annu. Rev. Food Sci. Technol. 4:327-346).

Flavonoids are pervasive plant secondary metabolites and have been of interest due to their multiple biological activities and benefits. Glycosylation has been used to modify these bioactive compounds to enhance solubility or the chemical and/or biological stability of aglycones. Typically, Leloir glycosyltransferases have been used to glycosylate flavonoids, but these enzymes provide low yields and require a nucleotide-sugar as a glycosyl donor. GH70 glucansucrases, which use sucrose as a glucose donor, have been reported to glucosylate various aglycones (Overwin et al., 2016, J. Biotechnol. 233:121-128; Li et al., 2021, Crit. Rev. Food Sci. Nutr. 1-21). However, this previous work generally has been drawn to synthesizing monoglycoside compounds, since further glycosylation might affect biological activities such as antioxidant activity.

### SUMMARY

In one embodiment, the present disclosure concerns a method of producing a food product/precursor, the method comprising: (a) providing a food product/precursor that comprises at least water, sucrose and a plant-based material, and (b) contacting the food product/precursor with at least: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, typically wherein at least one alpha-glucan is produced in the food product/precursor, whereby the food product/food precursor, after step (b), has reduced off-flavor as compared to the food product/precursor before step (b), and optionally has a reduced sugar content as compared to the food product/precursor before step (b).

In another embodiment, the present disclosure concerns a food product/precursor produced by a method herein.

In another embodiment, the present disclosure concerns method of glucosylating an isoflavone glycoside, the method comprising: providing a composition that comprises at least water, sucrose, an isoflavone glycoside, and a glucosyltransferase enzyme, wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, wherein at least one glucosylated form of the isoflavone glycoside is produced in the composition, and typically wherein at least one alpha-glucan is produced in the composition.

In another embodiment, the present disclosure concerns a composition, or a glucosylated isoflavone glycoside, produced by a method herein.

In another embodiment, the present disclosure concerns a composition comprising a glucosylated isoflavone glycoside, wherein the glucosylated isoflavone glycoside is produced by contacting an isoflavone glycoside with a glucosyltransferase enzyme in the presence of at least water and sucrose, wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, optionally wherein the composition is a food product/precursor.

### BRIEF DESCRIPTION OF THE SEQUENCES

TABLE A
Summary of Protein SEQ ID Numbers

| | Protein |
|---|---|
| Description | SEQ ID NO. |
| GTF 0768. Mature form of Leuconostoc | 1 |
| pseudomesenteroides protein, but with two additional | (1449 aa) |
| N-terminal amino acids. | |
| GTF 0768. Mature, wild type form of L. | 2 |
| pseudomesenteroides protein (US2016/0122445). | (1447 aa) |
| vGTFJ. Variant of GTF 6855 (SEQ ID NO: 5 below). | 3 |
| | (1341 aa) |
| Variant of GTF 6855 (SEQ ID NO: 5 below). | 4 |
| | (1341 aa) |
| GTF 6855, derivable from Streptococcus salivarius | 5 |
| SK126. The first 178 amino acids of the protein are | (1341 aa) |
| deleted compared to GENBANK Acc. No. | |
| ZP_04061500.1; a start methionine is included. | |
| GTF 7527, derivable from Streptococcus salivarius. | 6 |
| The first 178 amino acids of the protein are deleted | (1341 aa) |
| compared to GENBANK Acc. No. CAA77900.1; a | |
| start methionine is included. | |
| GTF 2678, derivable from Streptococcus salivarius | 7 |
| K12. The first 188 amino acids of the protein are | (1341 aa) |
| deleted compared to GENBANK Acc. No. | |
| EJO16940.1; a start methionine is included. | |
| GTF 2919, derivable from Streptococcus salivarius | 8 |
| PS4. The first 92 amino acids of the protein are | (1340 aa) |
| deleted compared to GENBANK Acc. No. | |
| EIC80898.1; a start methionine is included. | |
| GTF 2765, derivable from unknown Streptococcus sp. | 9 |
| C150. The first 193 amino acids of the protein are | (1340 aa) |
| deleted compared to GENBANK Acc. No. | |
| ZP_08047301.1; a start methionine is included. | |
| Wild type GTF corresponding to GTF 6855, | 10 |
| Streptococcus salivarius SK126 (GENBANK Acc. No. | (1518 aa) |
| ZP_04061500.1). | |
| GTF 0768. N-terminal-truncated form A of the mature | 11 |
| form of L. pseudomesenteroides protein (SEQ ID | (1423 aa) |
| NO: 2). The first 24 amino acids of the protein are | |
| deleted compared to SEQ ID NO: 2. | |
| GTF 0768. N-terminal-truncated form B of the mature | 12 |
| form of L. pseudomesenteroides protein (SEQ ID | (1375 aa) |
| NO: 2). The first 72 amino acids of the protein are | |
| deleted compared to SEQ ID NO: 2. | |
| GTF 0974, derivable from Streptococcus salivarius | 13 |
| 57.I. The first 187 amino acids of the protein are | (1392 aa) |
| deleted compared to GENBANK Acc. No. | |
| AEJ53088.1. | |
| GTF 0768. Alternative mature form of L. | 14 |
| pseudomesenteroides protein. A methionine replaces | (1449 aa) |
| the first amino acid compared to SEQ ID NO: 1. | |
| GTF 0768. Alternative mature form of L. | 15 |
| pseudomesenteroides protein, but with one additional | (1448 aa) |
| N-terminal amino acid compared to SEQ ID NO: 2. | |
| GTF 0768. N-terminal-truncated form C of the mature | 16 |
| form of L. pseudomesenteroides protein (SEQ ID | (1365 aa) |
| NO: 2). The first 82 amino acids of the protein are | |
| deleted compared to SEQ ID NO: 2. | |
| GTF 0768. N-terminal-truncated form D of the mature | 17 |
| form of L. pseudomesenteroides protein (SEQ ID | (1356 aa) |
| NO: 2). The first 91 amino acids of the protein are | |
| deleted compared to SEQ ID NO: 2. | |
| GTF 0768. N-terminal-truncated form E of the mature | 18 |
| form of L. pseudomesenteroides protein (SEQ ID | (1345 aa) |
| NO: 2). The first 102 amino acids of the protein are | |
| deleted compared to SEQ ID NO: 2. | |

### DETAILED DESCRIPTION

Unless otherwise disclosed, the terms “a”, “an” and “the” as used herein are intended to encompass one or more (i.e., at least one) of a referenced feature.

Where present, all ranges are inclusive and combinable, except as otherwise noted. For example, when a range of “1 to 5” (i.e., 1-5) is recited, the recited range should be construed as including ranges “1 to 4”, “1 to 3”, “1-2”, “1-2 & 4-5”, “1-3 & 5”, and the like.

The terms “alpha-glucan”, “alpha-glucan polymer” and the like are used interchangeably herein. An alpha-glucan is a polymer comprising glucose monomeric units linked together by alpha-glycosidic linkages. In typical embodiments, an alpha-glucan herein comprises at least about 80%, 81%, 82%, 83%, 84%, 85%, 86%, 87%, 88%, 89%, 90%, 91%, 92%, 93%, 94%, 95%, 96%, 97%, 98%, 99%, or 100% alpha-glycosidic linkages. Examples of alpha-glucan polymers herein include graft copolymers as presently disclosed, as well as alpha-1,3-glucan and alpha-1,6-glucan.

The terms “alpha-1,3-glucan”, “poly alpha-1,3-glucan”, “alpha-1,3-glucan polymer” and the like are used interchangeably herein. Alpha-1,3-glucan is a polymer comprising glucose monomeric units linked together by glycosidic linkages, wherein at least about 50% of the glycosidic linkages are alpha-1,3. Alpha-1,3-glucan in certain embodiments comprises at least about 90% or 95% alpha-1,3 glycosidic linkages. Most or all of the other linkages in alpha-1,3-glucan herein typically are alpha-1,6, though some linkages may also be alpha-1,2 and/or alpha-1,4. Alpha-1,3-glucan as presently disclosed can characterize an alpha-1,3-glucan side chain herein. In some aspects, alpha-1,3-glucan can characterize an alpha-1,3-glucan “homopolymer”, which is alpha-1,3-glucan that is not part of a dextran-alpha-1,3-glucan copolymer.

The terms “dextran”, “dextran polymer”, “dextran molecule”, “alpha-1,6-glucan” and the like herein refer to a water-soluble alpha-glucan comprising at least 50%, 60%, 70%, 80%, or 90% alpha-1,6 glycosidic linkages (with the balance of the linkages typically being alpha-1,3). Enzymes capable of synthesizing dextran from sucrose may be described as “dextransucrases” (EC 2.4.1.5). A “substantially linear” (“mostly linear”, and like terms) dextran has 5% or less branches, before being modified herein to have with alpha-1,3-glucan side chains. A “linear” dextran has no branches, before being modified herein to have alpha-1,3-glucan side chains. Branches, if present prior to modification of dextran with alpha-1,3-glucan side chains, can be short, being one (pendant) to three glucose monomers in length. Yet, in some aspects, dextran can be “dendritic”, which is a branched structure emanating from a core in which there are chains (containing mostly or all alpha-1,6-linkages) that iteratively branch from each other (e.g., a chain can be a branch from another chain, which in turn is a branch from another chain, and so on). Yet, in still some aspects, dextran is not dendritic, but has a branch-on-branch structure that does not emanate from a core. Dextran as used in a glucosyltransferase reaction herein for alpha-1,3-glucan synthesis (to produce a dextran-alpha-1,3-glucan copolymer) can optionally be characterized as a “primer” or “acceptor”. In some aspects, dextran can characterize a dextran “homopolymer”, which is dextran that is not part of a dextran-alpha-1,3-glucan copolymer.

The term “copolymer” herein refers to a polymer comprising at least two different types of alpha-glucan, such as dextran and alpha-1,3-glucan.

The terms “graft copolymer”, “branched copolymer” and the like herein generally refer to a copolymer comprising a “backbone” (or “main chain”) and one or more side chains branching from the backbone. The side chains are structurally distinct from the backbone.

Examples of graft copolymers herein are “dextran-alpha-1,3-glucan graft copolymers” (and like terms) that comprise a backbone comprising dextran, and one or more side chains of alpha-1,3-glucan. A backbone in some aspects can itself be a branched dextran as disclosed herein; the addition of alpha-1,3-glucan side chains to such a backbone (thereby forming a graft copolymer herein) can be, for example, via enzymatic extension from non-reducing ends presented by short branches (alpha-1,2, -1,3, or -1,4 branch, each typically comprised of a single glucose monomer; i.e., pendant glucose). Short branches (that can be enzymatically extended into an alpha-1,3-glucan side chain) can be present on an otherwise linear or mostly linear dextran, or can be present on a branching dextran. In some aspects, alpha-1,3-glucan can also be synthesized from non-reducing ends of dextran main chains, such as in embodiments in which the dextran backbone is linear or mostly linear, or embodiments in which the dextran backbone is branching (e.g., dendritic, or not dendritic [branches do not emanate from a core] but has branch-on-branch structure); such alpha-1,3-glucan is not, technically-speaking, a side chain to the dextran, but rather an extension from the dextran main chain(s).

The percent branching in an alpha-glucan herein refers to that percentage of all the linkages in the alpha-glucan that represent branch points. For example, the percent of alpha-1,3 branching in an alpha-glucan herein refers to that percentage of all the linkages in the glucan that represent alpha-1,3 branch points. Except as otherwise noted, linkage percentages disclosed herein are based on the total linkages of a glucan, or the portion of a glucan for which a disclosure specifically regards.

The terms “linkage”, “glycosidic linkage”, “glycosidic bond” and the like refer to the covalent bonds connecting the sugar monomers within a saccharide compound (oligosaccharides and/or polysaccharides). Examples of glycosidic linkages include 1,6-alpha-D-glycosidic linkages (herein also referred to as “alpha-1,6” linkages), 1,3-alpha-D-glycosidic linkages (herein also referred to as “alpha-1,3” linkages), 1,4-alpha-D-glycosidic linkages (herein also referred to as “alpha-1,4” linkages), and 1,2-alpha-D-glycosidic linkages (herein also referred to as “alpha-1,2” linkages). The glycosidic linkages of a glucan polymer herein can also be referred to as “glucosidic linkages”. Herein, “alpha-D-glucose” is referred to as “glucose”.

The glycosidic linkage profile of an alpha-glucan herein can be determined using any method known in the art. For example, a linkage profile can be determined using methods using nuclear magnetic resonance (NMR) spectroscopy (e.g., ¹³C NMR or ¹H NMR). These and other methods that can be used are disclosed in, for example, Food Carbohydrates: Chemistry, Physical Properties, and Applications (S. W. Cui, Ed., Chapter 3, S. W. Cui, Structural Analysis of Polysaccharides, Taylor & Francis Group LLC, Boca Raton, FL, 2005), which is incorporated herein by reference.

The “molecular weight” of an alpha-glucan herein can be represented as weight-average molecular weight (Mw) or number-average molecular weight (Mn), the units of which are in Daltons (Da) or grams/mole. In some aspects, molecular weight can be represented as DPw (weight average degree of polymerization) or DPn (number average degree of polymerization). DPw and DPn are calculated from the corresponding Mw or Mn, respectively, by dividing by the molar mass of one monomer unit M₁. In the case of glucan polymer, M₁=162.14. In some aspects, molecular weight can sometimes be provided as “DP” (degree of polymerization), which simply refers to the number of glucoses comprised within the alpha-glucan on an individual molecule basis. Various means are known in the art for calculating these various molecular weight measurements such as with high-pressure liquid chromatography (HPLC), size exclusion chromatography (SEC), or gel permeation chromatography (GPC).

The term “sucrose” herein refers to a non-reducing disaccharide composed of an alpha-D-glucose molecule and a beta-D-fructose molecule linked by an alpha-1,2-glycosidic bond. Sucrose is known commonly as table sugar. Sucrose can alternatively be referred to as “alpha-D-glucopyranosyl-(1→2)-beta-D-fructofuranoside”. “Alpha-D-glucopyranosyl” and “glucosyl” are used interchangeably herein.

The terms “sugar” or “sugars”, unless used to specifically refer to sucrose, refer to any monosaccharide, disaccharide, or oligosaccharide (e.g., ranging from DP3 to DP4, DP5, DP6, DP7, DP8, DP9, DP10, DP12, DP14, DP15, DP16, DP18, or DP20), such as those disclosed herein. Sugars herein typically are water-soluble.

The terms “glucosyltransferase”, “glucosyltransferase enzyme”, “GTF”, “glucansucrase” and the like are used interchangeably herein. The activity of a glucosyltransferase herein catalyzes the reaction of the substrate sucrose to make the products alpha-glucan and fructose. Other products (by-products) of a GTF reaction can include glucose, various soluble gluco-oligosaccharides, and leucrose. Wild type forms of glucosyltransferase enzymes generally contain (in the N-terminal to C-terminal direction) a signal peptide (which is typically removed by cleavage processes), a variable domain, a catalytic domain, and a glucan-binding domain. A glucosyltransferase herein is classified under the glycoside hydrolase family 70 (GH70) according to the CAZy (Carbohydrate-Active EnZymes) database (Cantarel et al., Nucleic Acids Res. 37:D233-238, 2009). The term “dextransucrase” (and like terms) can optionally be used to characterize a glucosyltransferase enzyme that produces dextran.

The term “glucosyltransferase catalytic domain” herein refers to the domain of a glucosyltransferase enzyme that provides alpha-glucan-synthesizing activity to a glucosyltransferase enzyme. A glucosyltransferase catalytic domain typically does not require the presence of any other domains to have this activity.

The terms “enzymatic reaction”, “glucosyltransferase reaction”, “glucan synthesis reaction”, “reaction composition”, “reaction formulation” and the like are used interchangeably herein and generally refer to a reaction that initially comprises water, sucrose, at least one active glucosyltransferase enzyme, and optionally other components. Components that can be further present in a glucosyltransferase reaction typically after it has commenced include fructose, glucose, leucrose, soluble gluco-oligosaccharides (e.g., DP2-DP7) (such may be considered as products or by-products, depending on the glucosyltransferase used), and/or insoluble alpha-glucan product(s) of DP8 or higher. It would be understood that certain glucan products, such as alpha-1,3-glucan with a degree of polymerization (DP) of at least 8 or 9, are water-insoluble and thus not dissolved in a glucan synthesis reaction. The term “under suitable reaction conditions” as used herein refers to reaction conditions that support conversion of sucrose to alpha-glucan product(s) via glucosyltransferase enzyme activity. It is during such a reaction that glucosyl groups originally derived from the input sucrose are enzymatically transferred and used in alpha-glucan polymer synthesis; glucosyl groups as involved in this process can thus optionally be referred to as the glucosyl component or moiety (or like terms) of a glucosyltransferase reaction.

The “yield” of an alpha-glucan product in a glucosyltransferase reaction in some aspects herein represents the molar yield based on the converted sucrose. The molar yield of an alpha-glucan product can be calculated based on the moles of the alpha-glucan product divided by the moles of the sucrose converted. Moles of converted sucrose can be calculated as follows: (mass of initial sucrose-mass of final sucrose)/molecular weight of sucrose [342 g/mol]. This molar yield calculation can be considered as a measure of selectivity of the reaction toward the alpha-glucan. In some aspects, the “yield” of an alpha-glucan product in a glucosyltransferase reaction can be based on the glucosyl component of the reaction. Such a yield (yield based on glucosyl) can be measured using the following formula:

Alpha-Glucan Yield=((IS/2−(FS/2+LE/2+GL+SO))/(IS/2−FS/2))×100%.

The fructose balance of a glucosyltransferase reaction can be measured to ensure that HPLC data, if applicable, are not out of range (90-110% is considered acceptable). Fructose balance can be measured using the following formula:

Fructose Balance=((180/342×(FS+LE)+FR)/(180/342×IS))×100%.

In the above two formulae, IS is [Initial Sucrose], FS is [Final Sucrose], LE is [Leucrose], GL is [Glucose], SO is [Soluble Oligomers] (gluco-oligosaccharides), and FR is [Fructose]; the concentrations of each foregoing substrate/product provided in double brackets are in units of grams/L and as measured by HPLC, for example.

The term “in situ” as used herein characterizes a glucosyltransferase reaction(s) that occurs inside a food product or precursor thereof and thereby produces alpha-glucan within the food product itself (or precursor). Such produced alpha-glucan (e.g., graft copolymer, alpha-1,3-glucan, and/or alpha-1,6-glucan) can be soluble or insoluble. While an alpha-1,3-glucan product is typically insoluble and an alpha-1,6-glucan product is typically soluble, a graft copolymer product can either be soluble or insoluble, in a food product/precursor herein. In situ production of alpha-glucan in a food product/precursor typically substitutes for adding alpha-glucan herein as an ingredient in food, though such addition can be performed if desired (e.g., to supplement the alpha-glucan produced in situ).

The terms “percent by volume”, “volume percent”, “vol %”, “v/v %” and the like are used interchangeably herein. The percent by volume of a solute in a solution can be determined using the formula: [(volume of solute)/(volume of solution)]×100%.

The terms “percent by weight”, “weight percentage (wt %)”, “weight-weight percentage (% w/w)” and the like are used interchangeably herein. Percent by weight refers to the percentage of a material on a mass basis as it is comprised in a composition, mixture, or solution.

The terms “weight/volume percent”, “w/V %” and the like are used interchangeably herein. Weight/volume percent can be calculated as: ((mass [g] of material)/(total volume [mL] of the material plus the liquid in which the material is placed))×100%. The material can be insoluble in the liquid (i.e., be a solid phase in a liquid phase, such as with a dispersion), or soluble in the liquid (i.e., be a solute dissolved in the liquid).

The terms “ingestible product” and “ingestible composition” are used interchangeably herein, and refer to any substance that, either alone or together with another substance, may be taken orally (i.e., by mouth), whether intended for consumption or not. Thus, an ingestible product includes food/beverage products. “Food/beverage products” refer to any edible product intended for consumption (e.g., for nutritional purposes) by humans or animals, including solids, semi-solids, or liquids. A “food” herein can optionally be referred to as a “foodstuff”, “food product”, or other like term, for example. Herein, unless otherwise disclosed, a beverage or other ingestible liquid is an example of a food product. While the present disclosure generally regards food and food precursors that are by definition intended for ingestion or eventual ingestion (food precursor first made into food before being eaten), the disclosure likewise regards other ingestible products (e.g., supplement, nutraceutical, pharmaceutical product) comprising in situ-produced alpha-glucan. A food precursor herein can be (i) a food as it exists before one or more processing steps (e.g., fermentation, aging, cooling/freezing, heating, baking, mixing) that render it to be a food product intended for direct consumption, and/or (ii) an ingredient for use in preparing a food product, for example. In some aspects, a food precursor can characterize a food product or ingredient as it exists before treatment with one or more GTF enzymes in a method herein.

The term “texture” as used herein in reference to a food product/precursor herein means the thickness of the food product/precursor and/or sensory perception of the food product/precursor by, for example, vision, touch, or oral/taste processing. An “improvement” in texture means an increase in thickness and/or an increase in the sensory perception. Unless otherwise noted, as used herein the “thickness” of a food product/precursor means the apparent viscosity extracted at shear rate of about 10-13 Hz (e.g., ~11.7 Hz) during a rheological analysis; an increase in apparent viscosity at such a shear rate indicates an increase in thickness. The apparent viscosity extracted at shear rate of about 230-270 Hz (e.g., ~249 Hz) during a rheological analysis is correlated to “mouthfeel”; an increase in apparent viscosity at such a shear rate indicates an increase in mouthfeel.

“Fermentation” and like terms herein as applied to food product/precursor refer to the conversion of carbohydrates in a food product/precursor into alcohol(s) and/or acid(s) through the action of one or more microorganisms (e.g., bacteria, yeast).

The terms “isoflavone glycoside”, “isoflavone monoglycoside” and like terms are used interchangeably herein, and refer to an isoflavone having a single glycoside group (glycosyl group such as glucosyl). Examples of isoflavone glycosides herein include daidzin (7-O-glucosyl-4′-hydroxyisoflavone), genistin (7-O-glucosyl-4′5-dihydroxyisoflavone) and glycitin (glycitein 7-O-beta-glucoside), which are monoglucosylated forms (have a single glucoside group) of the isoflavones daidzein, genistein and glycitein, respectively. Isoflavones such as daidzein, genistein and glycitein are examples of “aglucones” herein. A “glucosylated isoflavone glycoside” (and like terms) herein refers to an isoflavone glycoside that contains one or more (e.g., up to about 1, 2, 3, 4, 5, 6, 7, 8, 9, 10, or 11) glucosyl groups in addition to the glycosyl group (e.g., glucosyl group) already present in the isoflavone glycoside. It is believed that such one or more glucosyl groups can be within a linear chain of two or more glucosyl groups (i.e., a glucan), and/or in linkage from different sites (optionally in chains therefrom) of the isoflavone glycoside (e.g., extending from a glucoside group, or from another site of the isoflavone glycoside).

A composition herein that is “dry” or “dried” typically has less than 5, 4, 3, 2, 1, 0.5, or 0.1 wt % water comprised therein.

The terms “aqueous liquid”, “aqueous fluid”, “aqueous conditions”, “aqueous setting”, “aqueous system” and the like as used herein can refer to water or an aqueous solution. An “aqueous solution” herein can comprise one or more dissolved salts, where the maximal total salt concentration can be about 3.5 wt % in some embodiments. Although aqueous liquids herein typically comprise water as the only solvent in the liquid, an aqueous liquid can optionally comprise one or more other solvents (e.g., polar organic solvent) that are miscible in water. Thus, an aqueous solution can comprise a solvent having at least about 10 wt % water.

An “aqueous composition” herein has a liquid component that comprises about, or at least about, 10, 20, 30, 40, 50, 60, 70, 80, 90, 95, 99, or 100 wt % water, for example. Examples of aqueous compositions include mixtures, solutions, dispersions (e.g., suspensions, colloidal dispersions) and emulsions, for example.

An alpha-glucan herein that is “insoluble”, “aqueous-insoluble”, “water-insoluble” (and like terms) herein does not dissolve (or does not appreciably dissolve) in water or other aqueous conditions, optionally where the aqueous conditions are at a pH of 4-9 (e.g., pH 6-8) and/or a temperature of about 1 to 130° C. (e.g., 20-25° C.). In some aspects, less than 1.0 gram (e.g., no detectable amount) of an aqueous-insoluble alpha-glucan dissolves in 1000 milliliters of such aqueous conditions (e.g., water at 23° C.). In contrast, an alpha-glucan that is “soluble”, “aqueous-soluble”, “water-soluble” and the like appreciably dissolves under the above aqueous conditions.

The term “viscosity” as used herein refers to the resistance of a food product/precursor to deformation at a given rate. Viscosity may also be defined as a measure of the extent to which a fluid (aqueous or non-aqueous) resists a force tending to cause it to flow. Furthermore, viscosity can be defined as the shear stress resulting from an applied shear rate. Both dynamic and kinematic viscosity are meant by the term viscosity, as both parameters are directly correlated through the density of a food product/precursor. Various units of viscosity that can be used herein include centipoise (cP, cps) and Pascal-second (Pa·s), for example. A centipoise is one one-hundredth of a poise; one poise is equal to 0.100 kg·m⁻¹·s⁻¹.

As used herein, the term “polypeptide” is defined as a chain of amino acid residues, usually having a defined sequence. As used herein the term polypeptide is interchangeable with the terms “peptides” and “proteins”. Typical amino acids contained in polypeptides herein include (respective three- and one-letter codes shown parenthetically): alanine (Ala, A), arginine (Arg, R), asparagine (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), valine (Val, V).

The terms “sequence identity”, “identity” and the like as used herein with respect to polynucleotide or polypeptide sequences refer to the nucleic acid residues or amino acid residues in two sequences that are the same when aligned for maximum correspondence over a specified comparison window. Thus, “percentage of sequence identity”, “percent identity” and the like refer to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. It would be understood that, when calculating sequence identity between a DNA sequence and an RNA sequence, T residues of the DNA sequence align with, and can be considered “identical” with, U residues of the RNA sequence. For purposes of determining “percent complementarity” of first and second polynucleotides, one can obtain this by determining (i) the percent identity between the first polynucleotide and the complement sequence of the second polynucleotide (or vice versa), for example, and/or (ii) the percentage of bases between the first and second polynucleotides that would create canonical Watson and Crick base pairs.

Percent identity can be readily determined by any known method, including but not limited to those described in: 1) Computational Molecular Biology (Lesk, A. M., Ed.) Oxford University: NY (1988); 2) Biocomputing: Informatics and Genome Projects (Smith, D. W., Ed.) Academic: NY (1993); 3) Computer Analysis of Sequence Data, Part I (Griffin, A. M., and Griffin, H. G., Eds.) Humana: NJ (1994); 4) Sequence Analysis in Molecular Biology (von Heinje, G., Ed.) Academic (1987); and 5) Sequence Analysis Primer (Gribskov, M. and Devereux, J., Eds.) Stockton: NY (1991), all of which are incorporated herein by reference.

Preferred methods for determining percent identity are designed to give the best match between the sequences tested. Methods of determining identity and similarity are codified in publicly available computer programs, for example. Sequence alignments and percent identity calculations can be performed using the MEGALIGN program of the LASERGENE bioinformatics computing suite (DNASTAR Inc., Madison, WI), for example. Multiple alignment of sequences can be performed, for example, using the Clustal method of alignment which encompasses several varieties of the algorithm including the Clustal V method of alignment (described by Higgins and Sharp, CABIOS. 5:151-153 (1989); Higgins, D. G. et al., Comput. Appl. Biosci., 8:189-191 (1992)) and found in the MEGALIGN v8.0 program of the LASERGENE bioinformatics computing suite (DNASTAR Inc.). For multiple alignments, the default values can correspond to GAP PENALTY=10 and GAP LENGTH PENALTY=10. Default parameters for pairwise alignments and calculation of percent identity of protein sequences using the Clustal method can be KTUPLE=1, GAP PENALTY=3, WINDOW=5 and DIAGONALS SAVED=5. For nucleic acids, these parameters can be KTUPLE=2, GAP PENALTY=5, WINDOW=4 and DIAGONALS SAVED=4. Additionally, the Clustal W method of alignment can be used (described by Higgins and Sharp, CABIOS. 5:151-153 (1989); Higgins, D. G. et al., Comput. Appl. Biosci. 8:189-191 (1992); Thompson, J. D. et al, Nucleic Acids Research, 22 (22): 4673-4680, 1994) and found in the MEGALIGN v8.0 program of the LASERGENE bioinformatics computing suite (DNASTAR Inc.). Default parameters for multiple alignment (protein/nucleic acid) can be: GAP PENALTY=10/15, GAP LENGTH PENALTY=0.2/6.66, Delay Divergent Seqs (%)=30/30, DNA Transition Weight=0.5, Protein Weight Matrix=Gonnet Series, DNA Weight Matrix=IUB.

Various polypeptide amino acid sequences and polynucleotide sequences are disclosed herein as features of certain embodiments. Variants of these sequences that are at least about 70-85%, 85-90%, or 90%-95% identical to the sequences disclosed herein can be used or referenced. Alternatively, a variant amino acid sequence or polynucleotide sequence can have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity with a sequence disclosed herein. A variant amino acid sequence or polynucleotide sequence herein has the same function/activity of the disclosed sequence, or at least about 80%, 81%, 82%, 83%, 84%, 85%, 86%, 87%, 88%, 89%, 90%, 91%, 92%, 93%, 94%, 95%, 96%, 97%, 98%, 99%, 99.5% of the function/activity of the disclosed sequence. Any polypeptide amino acid sequence disclosed herein not beginning with a methionine or valine can typically further comprise at least a start-methionine or start-valine at the N-terminus of the amino acid sequence. In contrast, any polypeptide amino acid sequence disclosed herein beginning with a methionine or valine can optionally lack such a methionine or valine residue. In some aspects, any polypeptide amino acid sequence disclosed herein beginning with a methionine or valine can instead have, respectively, a valine or methionine as the first amino acid residue.

The terms “aligns with”, “corresponds with”, and the like can be used interchangeably herein. Some aspects herein relate to a glucosyltransferase comprising at least one amino acid substitution at a position corresponding with at least one particular amino acid residue of SEQ ID NO:10. An amino acid position of a glucosyltransferase or subsequence thereof (e.g., catalytic domain or catalytic domain plus glucan-binding domains) (can refer to such an amino acid position or sequence as a “query” position or sequence) can be characterized to correspond with a particular amino acid residue of SEQ ID NO:10 (can refer to such an amino acid position or sequence as a “subject” position or sequence) if (1) the query sequence can be aligned with the subject sequence (e.g., where an alignment indicates that the query sequence and the subject sequence [or a subsequence of the subject sequence] are at least about 30%, 40%, 50%, 60%, 70%, 80%, or 90% identical), and (2) if the query amino acid position directly aligns with (directly lines up against) the subject amino acid position in the alignment of (1). In general, one can align a query amino acid sequence with a subject sequence (SEQ ID NO:10 or a subsequence of SEQ ID NO:10) using any alignment algorithm, tool and/or software described disclosed herein (e.g., BLASTP, ClustalW, ClustalV, Clustal-Omega, EMBOSS) to determine percent identity. Just for further example, one can align a query sequence with a subject sequence herein using the Needleman-Wunsch algorithm (Needleman and Wunsch, J. Mol. Biol. 48:443-453, 1970) as implemented in the Needle program of the European Molecular Biology Open Software Suite (EMBOSS [e.g., version 5.0.0 or later], Rice et al., Trends Genet. 16:276-277, 2000). The parameters of such an EMBOSS alignment can comprise, for example: gap open penalty of 10, gap extension penalty of 0.5, EBLOSUM62 (EMBOSS version of BLOSUM62) substitution matrix.

The numbering of particular amino acid residues of SEQ ID NO:10 herein (e.g., Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, Glu-1450) is with respect to the full-length amino acid sequence of SEQ ID NO:10. The first amino acid (i.e., position 1, Met-1) of SEQ ID NO: 10 is at the start of the signal peptide. Unless otherwise disclosed, substitutions herein are in correspondence to the full-length amino acid sequence of SEQ ID NO:10 as reference sequence.

A “non-native glucosyltransferase” herein (“mutant”, “variant”, “modified” and like terms can likewise be used to describe such a glucosyltransferase) has at least one amino acid substitution at a position corresponding with a particular amino acid residue of SEQ ID NO:10 (SEQ ID NOs: 3 and 4 are examples of a non-native GTF). Such at least one amino acid substitution typically is in place of the amino acid residue(s) that normally (natively) occurs at the same position in the native counterpart (parent) of the non-native glucosyltransferase (i.e., although SEQ ID NO:10 is used as a reference for position, an amino acid substitution herein is with respect to the native counterpart of a non-native glucosyltransferase) (considered another way, when aligning the sequence of a non-native glucosyltransferase with SEQ ID NO:10, determining whether a substitution exists at a particular position does not depend in-and-of-itself on the respective amino acid residue in SEQ ID NO:10, but rather depends on what amino acid exists at the subject position within the native counterpart of the non-native glucosyltransferase). The amino acid normally occurring at the relevant site in the native counterpart glucosyltransferase often (but not always) is the same as (or conserved with) the particular amino acid residue of SEQ ID NO:10 for which the alignment is made. A non-native glucosyltransferase optionally can have other amino acid changes (mutations, deletions, and/or insertions) relative to its native counterpart sequence.

The term “isolated” means a substance (or process) in a form or environment that does not occur in nature. A non-limiting example of an isolated substance includes any non-naturally occurring substance such as a food product, food precursor, graft copolymer, and/or glucosylated isoflavone glycoside herein (as well as enzymatic reactions used to prepare these materials). It is believed that the embodiments disclosed herein are synthetic/man-made (could not have been made except for human intervention/involvement), and/or have properties that are not naturally occurring.

The term “increased” as used herein can refer to a quantity or activity that is at least about 1%, 2%, 3%, 4%, 5%, 6%, 7%, 8%, 9%, 10%, 11%, 12%, 13%, 14%, 15%, 16%, 17%, 18%, 19%, 20%, 50%, 100%, or 200% more than the quantity or activity for which the increased quantity or activity is being compared. The terms “increased”, “elevated”, “enhanced”, “greater than”, “improved” and the like are used interchangeably herein.

Some embodiments of the present disclosure concern a method of producing a food product/precursor. Such a method can comprise:
(a) providing a food product/precursor that comprises at least water, sucrose and a plant-based material, and
(b) contacting the food product/precursor with at least:
   (i) a glucosyltransferase enzyme that synthesizes alpha-1,6-glucan, wherein at least about 50% of the glycosidic linkages of the alpha-1,6-glucan are alpha-1,6 linkages, and/or
   (ii) a glucosyltransferase enzyme that synthesizes alpha-1,3-glucan, wherein at least about 50% of the glycosidic linkages of the alpha-1,3-glucan are alpha-1,3 linkages,
   typically wherein at least one alpha-glucan is produced in the food product/precursor,
whereby the food product/food precursor, after step (b), has reduced off-flavor as compared to the food product/precursor before step (b),
and optionally has a reduced sugar content as compared to the food product/precursor before step (b).

Step (b) of producing a food product/precursor can comprise contacting a food product/precursor with at least:
(i) a glucosyltransferase (GTF) enzyme that synthesizes alpha-1,6-glucan, wherein at least about 50% of the glycosidic linkages of the alpha-1,6-glucan are alpha-1,6 linkages, and/or
(ii) a GTF enzyme that synthesizes alpha-1,3-glucan, wherein at least about 50% of the glycosidic linkages of the alpha-1,3-glucan are alpha-1,3 linkages.

In some aspects, a GTF enzyme (dextransucrase) that synthesizes alpha-1,6-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO:1, 2, 11, 12, 14, 15, 16, 17, or 18 (GTF 0768), and have GTF activity. Yet, in some aspects, a GTF enzyme that synthesizes alpha-1,6-glucan can be as disclosed in any of U.S. Patent Appl. Publ. Nos. 2017/0218093, 2018/0282385, 2018/0291311, or 2016/0122445, which are each incorporated herein by reference. For example, the GTF identified as GTF 8117 (SEQ ID NO:30), GTF 6831 (SEQ ID NO:32), or GTF 5604 (SEQ ID NO:33) in US2018/0282385 can be used, or the GTF identified as GTF 2919 (SEQ ID NO:5), GTF 2918 (SEQ ID NO:9), GTF 2920 (SEQ ID NO: 13), or GTF 2921 (SEQ ID NO:17) in US2016/0122445 can be used, or a GTF comprising an amino acid sequence that is about 100% identical to, or at least about 90%, 91%, 92%, 93%, 94%, 95%, 96%, 97%, 98%, 98.5%, 99%, or 99.5% identical to, the amino acid sequence of any of these GTF enzymes (and having GTF activity) can be used.

A dextransucrase herein is capable of producing dextran comprising about, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% alpha-1,6 glycosidic linkages, for example. Such a percent alpha-1,6 linkage profile takes into account the total of all linkages in the dextran (main chains of alpha-1,6 glucan and, if present, branch portions therefrom). Dextran as disclosed elsewhere herein such as in a homopolymer or graft-copolymer can have any of the foregoing linkage profiles, for example.

A dextransucrase herein is capable of producing dextran having a weight-average molecular weight (Mw) of about, at least about, or less than about, 1000, 2500, 5000, 7500, 10000, 25000, 50000, 75000, 100000, 150000, 200000, 250000, 500000, 750000, 1000000, 1000-10000, 1000-100000, 1000-1000000, 10000-100000, 10000-1000000, or 100000-1000000 Daltons, for example. In some aspects, the Mw is about, at least about, or less than about, 1, 2, 3, 4, 5, 6, 7, 8, 9, 10, 20, 30, 40, 50, 60, 70, 80, 90, 100, 110, 120, 130, 140, 150, 160, 170, 180, 190, 200, 10-50, 10-70, 10-80, 10-100, 10-120, 10-130, 10-150, 10-200, 25-50, 25-70, 25-80, 25-100, 25-120, 25-130, 25-150, 25-200, 50-70, 50-80, 50-100, 50-120, 50-130, 50-150, 50-200, 70-80, 70-100, 70-120, 70-130, 70-150, 70-200, 80-100, 80-120, 80-130, 80-150, 80-200, 100-120, 100-130, 100-150, 100-200, 120-130, 120-150, 120-200, 130-150, or 130-200 million Daltons, for example. Dextran as disclosed elsewhere herein such as in a homopolymer or graft-copolymer can have any of the foregoing molecular weight profiles, for example.

In some aspects, a GTF enzyme that synthesizes alpha-1,3-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO: 2, 4, 6, 8, 10, 12, 14, 16, 18, 20, 26, 28, 30, 34, or 59, or amino acid residues 55-960 of SEQ ID NO:4, residues 54-957 of SEQ ID NO:65, residues 55-960 of SEQ ID NO: 30, residues 55-960 of SEQ ID NO:28, or residues 55-960 of SEQ ID NO:20, and have GTF activity; these amino acid sequences are disclosed in U.S. Patent Appl. Publ. No. 2019/0078063, which is incorporated herein by reference. It is noted that such a GTF enzyme comprising SEQ ID NO:2, 4, 8, 10, 14, 20, 26, 28, 30, 34, or amino acid residues 55-960 of SEQ ID NO:4, residues 54-957 of SEQ ID NO:65, residues 55-960 of SEQ ID NO:30, residues 55-960 of SEQ ID NO:28, or residues 55-960 of SEQ ID NO: 20, can synthesize alpha-glucan comprising at least about 90% (~100%) alpha-1,3 linkages. A GTF enzyme that synthesizes alpha-1,3-glucan in some aspects can be that identified as GTF 0974 (SEQ ID NO:13 herein, SEQ ID NO:110 in US2018/0291311), or a GTF comprising an amino acid sequence that is about 100% identical to, or at least about 90%, 91%, 92%, 93%, 94%, 95%, 96%, 97%, 98%, 98.5%, 99%, or 99.5% identical to, the foregoing amino acid sequence of GTF 0974 (and having GTF activity). Any of the foregoing GTF enzyme amino acid sequences can be modified as described herein to increase product yield, modify product molecular weight, and/or enhance GTF performance and/or stability.

A GTF enzyme for producing alpha-1,3-glucan herein can, in some aspects, synthesize alpha-1,3-glucan at a yield of at least about 40%, 45%, 50%, 55%, 60%, 65%, 70%, 75%, 80%, 85%, 90%, 91%, 92%, 93%, 94%, 95%, or 96%. Yield in some aspects can be measured based on the glucosyl component of the reaction, and/or as measured using HPLC or NIR spectroscopy. Yield can be achieved in a reaction conducted for about 16-24 hours (e.g., ~20 hours), for example. Examples of such a GTF enzyme are those having an amino acid sequence modified such that the enzyme produces more products (alpha-1,3-glucan and fructose), and less by-products (e.g., glucose, oligosaccharides such as leucrose), from a given amount of sucrose substrate. For example, one, two, three, four, or more amino acid residues of the catalytic domain of an alpha-1,3-glucan-producing GTF herein can be modified/substituted to obtain a GTF enzyme that produces more products. Examples of a suitable modified GTF enzyme are disclosed in Tables 3-7 of U.S. Patent Appl. Publ. No. 2019/0078063. A modified GTF enzyme, for example, can comprise one or more amino acid substitutions corresponding with those in Tables 3-7 (ibid.) that is/are associated with an alpha-1,3-glucan yield of at least 40% (the position numbering of such at least one substitution corresponds with the position numbering of SEQ ID NO:62 as disclosed in U.S. Patent Appl. Publ. No. 2019/0078063). A set of amino acid modifications as listed in Tables 6 or 7 (ibid.) can be used, for example.

The amino acid sequence of a GTF enzyme for alpha-1,3-glucan synthesis in some aspects has been modified such that the enzyme produces alpha-1,3-glucan with a molecular weight (DPw) that is lower than the molecular weight of alpha-1,3-glucan produced by its corresponding parent GTF. Examples of a suitable modified GTF enzyme are disclosed in Tables 3 and 4 of U.S. Patent Appl. Publ. No. 2019/0276806, which is incorporated herein by reference. A modified GTF enzyme, for example, can comprise one or more amino acid substitutions corresponding with those in Tables 3 and/or 4 (ibid.) that is/are associated with an alpha-1,3-glucan product molecular weight that is at least 5% less than the molecular weight of alpha-1,3-glucan produced by parent enzyme (the position numbering of such at least one substitution corresponds with the position numbering of SEQ ID NO:62 as disclosed in U.S. Patent Appl. Publ. No. 2019/0276806). A set of amino acid modifications as listed in Table 4 (ibid.) can be used, for example.

The amino acid sequence of a GTF enzyme for alpha-1,3-glucan synthesis in some aspects has been modified such that the enzyme produces alpha-1,3-glucan with a molecular weight (DPw) that is higher than the molecular weight of alpha-1,3-glucan produced by its corresponding parent GTF. Examples of a suitable modified GTF enzyme are disclosed in Tables 3, 4 and 5 of U.S. Patent Appl. Publ. No. 2019/0078062, which is incorporated herein by reference. A modified GTF enzyme, for example, can comprise one or more amino acid substitutions corresponding with those in Tables 3, 4 and/or 5 (ibid.) that is/are associated with an alpha-1,3-glucan product molecular weight that is at least 5% higher than the molecular weight of alpha-1,3-glucan produced by parent enzyme (the position numbering of such at least one substitution corresponds with the position numbering of SEQ ID NO:62 as disclosed in U.S. Patent Appl. Publ. No. 2019/0078062). A set of amino acid modifications as listed in Table 5 (ibid.) can be used, for example.

In some aspects, a modified GTF for alpha-1,3-glucan synthesis (i) comprises at least one amino acid substitution or a set of amino acid substitutions (as described above regarding yield or molecular weight), and (ii) comprises or consists of a GTF catalytic domain that is at least about 90%, 91%, 92%, 93%, 94%, 95%, 96%, 97%, 98%, 99%, or 99.5% identical to amino acid residues 55-960 of SEQ ID NO:4, amino acid residues 54-957 of SEQ ID NO:65, amino acid residues 55-960 of SEQ ID NO:30, amino acid residues 55-960 of SEQ ID NO:28, or amino acid residues 55-960 of SEQ ID NO: 20 (each of these sequences as disclosed in U.S. Patent Appl. Publ. No. 2019/0078063, which is incorporated herein by reference). Each of these subsequences are the approximate catalytic domains of each respective reference sequence, and produce alpha-1,3-glucan comprising at least about 50% (e.g., ≥90% or ≥95%) alpha-1,3 linkages. In some aspects, a modified GTF (i) comprises at least one amino acid substitution or a set of amino acid substitutions (as described above), and (ii) comprises or consists of an amino acid sequence that is at least about 90%, 91%, 92%, 93%, 94%, 95%, 96%, 97%, 98%, 99%, or 99.5% identical to SEQ ID NO:62 or a subsequence thereof such as SEQ ID NO:4 (without start methionine thereof) or positions 55-960 of SEQ ID NO:4 (approximate catalytic domain) (each of these sequences as disclosed in U.S. Patent Appl. Publ. No. 2019/0078063).

In the present disclosure, SEQ ID NOs: 5, 6, 7, 8, 9 and 10 (Table A) are the same amino acid sequences as, respectively, SEQ ID NOs: 4, 65, 30, 28, 20 and 62 as disclosed in U.S. Patent Appl. Publ. No. 2019/0078063. Thus, each of presently disclosed SEQ ID NOs: 5, 6, 7, 8, 9 and 10 can be used in any of the disclosed aspects, as appropriate. For example, a GTF enzyme that synthesizes alpha-1,3-glucan herein can comprise an amino acid sequence that is about 100% identical to, or at least about 90%, 91%, 92%, 93%, 94%, 95%, 96%, 97%, 98%, 98.5%, 99%, or 99.5% identical to, SEQ ID NO:5, 6, 7, 8, 9, or 10, or amino acid residues 55-960 of SEQ ID NO:5, residues 54-957 of SEQ ID NO:6, residues 55-960 of SEQ ID NO:7, residues 55-960 of SEQ ID NO: 8, or residues 55-960 of SEQ ID NO:9. Any of these sequences can be modified as described herein to affect alpha-1,3-glucan yield and/or molecular weight and/or stability, for example.

In some aspects, a GTF enzyme for alpha-1,3-glucan synthesis has been modified such that the enzyme has enhanced performance and/or stability benefit(s). Such a modification can be, for example, by having one, two, three, four, five, six, seven, eight, nine, ten, or more amino acid substitutions as compared to a corresponding parent GTF enzyme (e.g., a wild type mature GTF or active subsequence thereof such as a catalytic domain). Exemplary performance and/or stability benefits herein include one or more of increased thermal stability, increased storage stability, increased solubility, better pH profile, increased specific activity, modified substrate specificity, modified substrate binding, modified pH-dependent activity, modified pH-dependent stability, increased oxidative stability, increased expression, and/or increased glucan product yield (and/or decreased byproduct [e.g., leucrose] yield). In some aspects, a performance benefit is realized at a relatively low temperature (e.g., <5° C.) or at a relatively high temperature (e.g., >40° C.). An increase in any of the foregoing features can be by about, or at least about, 5%, 10%, 15%, 20%, 25%, or 30%, for example, as compared to the respective activity of a parent GTF enzyme that has not been modified.

Some examples of modified alpha-1,3-glucan-producing GTF enzymes herein having enhanced performance and/or stability benefit(s) comprise or consist of SEQ ID NO: 3 (vGTFJ) or 4. It is noted that SEQ ID NOs: 3 and 4 are both derivable from SEQ ID NO: 5 (GTF 6855), for example (e.g., SEQ ID NO:5 can be a backbone for making substitutions to render SEQ ID NOs: 3 and 4).

It is noted that, as compared to SEQ ID NO:5, SEQ ID NO:3 has the following amino acid substitutions: Tyr-8-Asn (i.e., position 8 is substituted with Asn, in place of Tyr, as compared to SEQ ID NO:5), Val-9-Ala, Leu-336-Tyr, Gln-411-Leu, Phe-430-Tyr, Lys-448-Ala, Arg-564-Ser, Thr-1254-Gln and Glu-1273-Phe (aside from having a valine at position 1). The positions of each of these substitutions correspond, respectively, with positions Tyr-185, Val-186, Leu-513, Gln-588, Phe-607, Lys-625, Arg-741, Thr-1431 and Glu-1450 of SEQ ID NO:10, which is used as a reference sequence herein.

It is noted that, as compared to SEQ ID NO:5, SEQ ID NO:4 has the following amino acid substitutions: Leu-336-Tyr, Phe-430-Tyr, Ile-431-Val, Lys-448-Ala, Arg-564-Ser, Val-1011-Glu, Lys-1150-His, Glu-1155-Ala, Asp-1241-Lys, Ala-1242-Glu, Ser-1243-Gly, Thr-1244-Ser, Arg-1247-Leu and Leu-1248-Val (aside from having a valine at position 1). The positions of each of these substitutions correspond, respectively, with positions Leu-513, Phe-607, Ile-608, Lys-625, Arg-741, Val-1188, Lys-1327, Glu-1332, Asp-1418, Ala-1419, Ser-1420, Thr-1421, Arg-1424 and Leu-1425 of SEQ ID NO:10, which is used as a reference sequence herein.

In some aspects of the present disclosure, a modified GTF enzyme can comprise or consist of an amino acid sequence that is at least about 90%, 91%, 92%, 93%, 94%, 95%, 96%, 97%, 98%, 99%, or 99.5% identical to SEQ ID NO:3, and have one or more of (or all of) the following amino acid residues: 8-Asn, 9-Ala, 336-Tyr, 411-Leu, 430-Tyr, 448-Ala, 564-Ser, 1254-Gln, and/or 1273-Phe. The valine at position 1 of SEQ ID NO:3 in any of the foregoing aspects can optionally instead be a methionine, or can be deleted.

In some aspects of the present disclosure, a modified GTF enzyme can comprise or consist of an amino acid sequence that is at least about 90%, 91%, 92%, 93%, 94%, 95%, 96%, 97%, 98%, 99%, or 99.5% identical to SEQ ID NO:4, and have one or more of (or all of) the following amino acid residues: 336-Tyr, 430-Tyr, 431-Val, 448-Ala, 564-Ser, 1011-Glu, 1150-His, 1155-Ala, 1241-Lys, 1242-Glu, 1243-Gly, 1244-Ser, 1247-Leu, and/or 1248-Val. The valine at position 1 of SEQ ID NO:4 in any of the foregoing aspects can optionally instead be a methionine, or can be deleted.

Any of the amino acids listed above in aspects related to SEQ ID NOs: 3 and 4 can optionally instead be another amino acid selected from Table B based on amino acid conservation.

TABLE B
Amino Acid Conservation

| For Amino | | |
|---|---|---|
| Acid | Code | Replace with any of: |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, b-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, |
| | | Val, D-Val |
| Phenylalanine | F | D-Phe, Tyr, D-Thr, L-Dopa, His, D-His, Trp, |
| | | D-Trp, Trans-3,4, or 5-phenylproline, |
| | | cis-3,4, or 5-phenylproline |
| Proline | P | D-Pro, L-I-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

In some aspects, a modified alpha-1,3-glucan-producing GTF enzyme having enhanced performance and/or stability benefit(s) comprises one, two, three, four, five, six, seven, eight, nine, ten, or more amino acid substitution(s) at a position(s) corresponding with amino acid residue(s) Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, and/or Glu-1450 of SEQ ID NO:10. For example, a modified alpha-1,3-glucan-producing GTF enzyme can comprise amino acid substitutions at positions corresponding with the following amino acid residues of SEQ ID NO: 10:
(i) Tyr-185, Val-186, Leu-513, Gln-588, Phe-607, Lys-625, Arg-741, Thr-1431 and/or Glu-1450 (these positions correspond to those in the GTF of SEQ ID NO:3);
(ii) Leu-513, Phe-607, Ile-608, Lys-625, Arg-741, Val-1188, Lys-1327, Glu-1332, Asp-1418, Ala-1419, Ser-1420, Thr-1421, Arg-1424, and/or Leu-1425 (these positions correspond to those in the GTF of SEQ ID NO:4);
(iii) Tyr-185, Val-186, Lys-625, Thr-1431, and/or Glu-1450;
(iv) Ile-608, Lys-625, Lys-1327, Glu-1332, Asp-1418, Ala-1419, Ser-1420, Thr-1421, Arg-1424, and/or Leu-1425;
(v) Leu-513, Gln-588, Phe-607, Lys-625, and/or Arg-741;
(vi) Leu-513, Phe-607, Ile-608, Lys-625, and/or Arg-741; and/or
(vii) Leu-513, Phe-607, Lys-625, and/or Arg-741.

In some aspects regarding a modified alpha-1,3-glucan-producing GTF enzyme having enhanced performance and/or stability benefit(s),
(a) the amino acid substitution at a position corresponding with amino acid residue Tyr-185 of SEQ ID NO:10 can be with an Asn residue, or any residue that is conserved with Asn (e.g., Table B);
(b) the amino acid substitution at a position corresponding with amino acid residue Val-186 of SEQ ID NO:10 can be with an Ala residue, or any residue that is conserved with Ala (e.g., Table B);
(c) the amino acid substitution at a position corresponding with amino acid residue Leu-513 of SEQ ID NO:10 can be with a Tyr, Phe, or Trp residue, or any residue that is conserved with Tyr, Phe, or Trp (e.g., Table B);
(d) the amino acid substitution at a position corresponding with amino acid residue Gln-588 of SEQ ID NO:10 can be with a Leu residue, or any residue that is conserved with Leu (e.g., Table B);
(e) the amino acid substitution at a position corresponding with amino acid residue Ile-608 of SEQ ID NO: 10 can be with a Val or Tyr residue, or any residue that is conserved with Val or Tyr (e.g., Table B);
(f) the amino acid substitution at a position corresponding with amino acid residue Lys-625 of SEQ ID NO: 10 can be with an Ala residue, or any residue that is conserved with Ala (e.g., Table B);
(g) the amino acid substitution at a position corresponding with amino acid residue Arg-741 of SEQ ID NO:10 can be with a Ser residue, or any residue that is conserved with Ser (e.g., Table B);
(h) the amino acid substitution at a position corresponding with amino acid residue Val-1188 of SEQ ID NO:10 can be with a Glu residue, or any residue that is conserved with Glu (e.g., Table B);
(i) the amino acid substitution at a position corresponding with amino acid residue Lys-1327 of SEQ ID NO:10 can be with a His residue, or any residue that is conserved with His (e.g., Table B);
(j) the amino acid substitution at a position corresponding with amino acid residue Glu-1332 of SEQ ID NO:10 can be with an Ala residue, or any residue that is conserved with Ala (e.g., Table B);
(k) the amino acid substitution at a position corresponding with amino acid residue Asp-1418 of SEQ ID NO:10 can be with a Lys residue, or any residue that is conserved with Lys (e.g., Table B);
(l) the amino acid substitution at a position corresponding with amino acid residue Ala-1419 of SEQ ID NO:10 can be with a Glu residue, or any residue that is conserved with Glu (e.g., Table B);
(m) the amino acid substitution at a position corresponding with amino acid residue Ser-1420 of SEQ ID NO: 10 can be with a Gly residue, or any residue that is conserved with Gly (e.g., Table B);
(n) the amino acid substitution at a position corresponding with amino acid residue Thr-1421 of SEQ ID NO:10 can be with a Ser residue, or any residue that is conserved with Ser (e.g., Table B);
(o) the amino acid substitution at a position corresponding with amino acid residue Arg-1424 of SEQ ID NO:10 can be with a Leu residue, or any residue that is conserved with Leu (e.g., Table B);
(p) the amino acid substitution at a position corresponding with amino acid residue Leu-1425 of SEQ ID NO:10 can be with a Val residue, or any residue that is conserved with Val (e.g., Table B);
(q) the amino acid substitution at a position corresponding with amino acid residue Thr-1431 of SEQ ID NO:10 can be with a Gln residue, or any residue that is conserved with Gln (e.g., Table B); and/or
(r) the amino acid substitution at a position corresponding with amino acid residue Glu-1450 of SEQ ID NO:10 can be with a Phe residue, or any residue that is conserved with Phe (e.g., Table B).

Although it is believed that a modified alpha-1,3-glucan-producing GTF enzyme in some aspects need only have a catalytic domain, the modified GTF can be comprised within a larger amino acid sequence. For example, a catalytic domain may be linked at its C-terminus to a glucan-binding domain, and/or linked at its N-terminus to a variable domain and/or signal peptide.

Although amino acid substitutions in a modified alpha-1,3-glucan-producing GTF enzyme are generally disclosed in some aspects with respect to corresponding positions in SEQ ID NO:10, such substitutions can alternatively be stated simply with respect to its/their position number in the amino acid sequence used to produce the modified GTF itself (e.g., SEQ ID NO:5 [optionally without start methionine thereof] or positions 55-960 of SEQ ID NO:5 [approximate catalytic domain]), as convenience may dictate. Such can be done simply by aligning the amino acid sequence with SEQ ID NO:10 and identifying the position number(s) of interest in the amino acid sequence based on its/their direct alignment with the corresponding position(s) in SEQ ID NO:10.

An alpha-1,3-glucan-producing GTF herein is capable of producing alpha-1,3-glucan comprising about, or at least about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% alpha-1,3-glycosidic linkages, for example. Alpha-1,3-glucan as disclosed elsewhere herein such as in a homopolymer or graft-copolymer can have any of the foregoing linkage profiles, for example.

An alpha-1,3-glucan-producing GTF herein is capable of producing alpha-1,3-glucan with a DPw, DPn, or DP of about, at least about, or less than about, 11, 12, 15, 20, 25, 30, 35, 40, 45, 50, 55, 60, 65, 70, 75, 80, 85, 90, 95, 100, 150, 200, 300, 400, 500, 600, 700, 800, 900, 1000, 1100, 1200, 1300, 1400, 1500, 1600, or 1650, for example. DPw, DPn, or DP can optionally be expressed as a range between any two of these values. Merely as examples, the DPw, DPn, or DP can be about 100-1650, 200-1650, 300-1650, 400-1650, 500-1650, 600-1650, 700-1650, 100-1250, 200-1250, 300-1250, 400-1250, 500-1250, 600-1250, 700-1250, 100-1000, 200-1000, 300-1000, 400-1000, 500-1000, 600-1000, 700-1000, 100-900, 200-900, 300-900, 400-900, 500-900, 600-900, 700-900, 11-25, 12-25, 11-22, 12-22, 11-20, 12-20, 20-300, 20-200, 20-150, 20-100, 20-75, 30-300, 30-200, 30-150, 30-100, 30-75, 50-300, 50-200, 50-150, 50-100, 50-75, 75-300, 75-200, 75-150, 75-100, 100-300, 100-200, 100-150, 150-300, 150-200, or 200-300. Alpha-1,3-glucan as disclosed elsewhere herein such as in a homopolymer or graft-copolymer can have any of the foregoing molecular weight profiles, for example.

In some aspects, a GTF enzyme can be any as disclosed herein and include 1-300 (or any integer there between [e.g., 10, 15, 20, 25, 30, 35, 40, 45, or 50]) residues on the N-terminus and/or C-terminus. Such additional residues can be from a corresponding wild type sequence from which the GTF enzyme is derivable, or can be a heterologous sequence such as an epitope tag (at either N- or C-terminus) or a heterologous signal peptide (at N-terminus), for example. A GTF enzyme herein typically lacks an N-terminal signal peptide; such an enzyme can optionally be characterized as being mature if its signal peptide was removed during a secretion process.

A GTF enzyme herein can typically be derived from bacteria. Examples of bacterial GTF enzymes are those derived from a Streptococcus species, Leuconostoc species, or Lactobacillus species. Examples of Streptococcus species include S. salivarius, S. sobrinus, S. dentirousetti, S. downei, S. mutans, S. oralis, S. gallolyticus and S. sanguinis. Examples of Leuconostoc species include L. mesenteroides, L. amelibiosum, L. argentinum, L. carnosum, L. citreum, L. cremoris, L. dextranicum and L. fructosum. Examples of Lactobacillus species include L. acidophilus, L. delbrueckii, L. helveticus, L. salivarius, L. casei, L. curvatus, L. plantarum, L. sakei, L. brevis, L. buchneri, L. fermentum and L. reuteri.

A GTF enzyme herein can be prepared by fermentation of an appropriately engineered microbial strain, for example. Recombinant enzyme production by fermentation can be done, for example, using microbial species such as E. coli, Bacillus strains (e.g., B. subtilis), Ralstonia eutropha, Pseudomonas fluorescens, Saccharomyces cerevisiae, Pichia pastoris, Hansenula polymorpha, and species of Aspergillus (e.g., A. awamori) and Trichoderma (e.g., T. reesei) (e.g., see Adrio and Demain, Biomolecules 4:117-139, 2014, which is incorporated herein by reference). A nucleotide sequence encoding a GTF amino acid sequence is typically linked to a heterologous promoter sequence to create an expression cassette for the enzyme, and/or is codon-optimized accordingly. Such an expression cassette can be incorporated in a suitable plasmid or integrated into the microbial host chromosome. The expression cassette can include a transcriptional terminator nucleotide sequence following the amino acid coding sequence. The expression cassette can also include, between the promoter sequence and GTF amino acid coding sequence, a nucleotide sequence encoding a signal peptide (e.g., heterologous signal peptide) that is designed for direct secretion of the GTF enzyme. At the end of fermentation, cells can be ruptured accordingly (generally when a signal peptide for secretion is not employed) and the GTF enzyme can be isolated using methods such as precipitation, filtration, and/or concentration. Alternatively, a lysate or extract comprising a GTF can be used without further isolation. If the GTF was secreted (i.e., it is present in the fermentation broth), it can optionally be used as isolated from, or as comprised in, the fermentation broth. The activity of a GTF enzyme can be confirmed by biochemical assay, such as measuring its conversion of sucrose to glucan polymer.

Alpha-glucan produced in step (b) of producing a food product/precursor in some aspects comprises a graft copolymer comprising:
(i) an alpha-1,6-glucan (dextran) backbone, wherein at least about 50% of the glycosidic linkages of the alpha-1,6-glucan (dextran) backbone are alpha-1,6 linkages, and
(ii) at least one alpha-1,3-glucan side chain, wherein at least about 50% of the glycosidic linkages of the alpha-1,3-glucan chain are alpha-1,3 linkages.

Such a graft copolymer can be aqueous-soluble or aqueous-insoluble. Dextran backbone of an alpha-glucan graft copolymer herein can be dextran as presently disclosed, for example, or can be as disclosed (e.g., molecular weight, linkage/branching profile, production method) in U.S. Patent Appl. Publ. Nos. 2016/0122445, 2017/0218093, 2018/0282385, 2020/0165360, or 2019/0185893, which are each incorporated herein by reference. In some aspects, a dextran backbone (before being integrated into a graft copolymer) has been alpha-1,2- and/or alpha-1,3-branched; the percent alpha-1,2 and/or alpha-1,3 branching of a backbone of a graft copolymer herein can be about, at least about, or less than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45%, 50%, 2-25%, 2-20%, 2-15%, 2-10%, 5-25%, 5-20%, 5-15%, 5-10%, 7-13%, 8-12%, 9-11%, 10-25%, 10-20%, or 10-15%, for example. Alpha-1,3-glucan side chain(s) of an alpha-glucan graft copolymer herein can be alpha-1,3-glucan as presently disclosed, for example, or can be as disclosed (e.g., molecular weight, linkage profile), in U.S. Pat. Nos. 7,000,000, 8,871,474, 10,301,604, or 10,260,053, or U.S. Patent Appl. Publ. Nos. 2019/0112456, 2019/0078062, 2019/0078063, 2018/0340199, 2018/0021238, 2018/0273731, 2017/0002335, 2015/0232819, 2015/0064748, 2020/0165360, 2020/0131281, or 2019/0185893, which are each incorporated herein by reference.

One, two, three, or more different GTF enzymes that synthesize alpha-1,6-glucan herein can be used, for example, in step (b) of producing a food product/precursor. Similarly, one, two, three, or more different GTF enzymes that synthesize alpha-1,3-glucan herein can be used, for example. In some aspects, an alpha-1,6-glucan-producing GTF(s) can be added to (made to contact) a food product/precursor before adding an alpha-1,3-glucan-producing GTF(s), while in some aspects both these types of GTF enzymes can be added at about the same time (simultaneously). Still, in some aspects, an alpha-1,3-glucan-producing GTF(s) can be added to a food product/precursor before adding an alpha-1,6-glucan-producing GTF(s). Still, in some aspects, a dextran as disclosed herein, but produced exogenously to the food product/precursor, can be added as an ingredient to a food product/precursor to which an alpha-1,3-glucan-producing GTF has already been added or will be added. While not being held to any particular theory, it is believed that addition of at least one alpha-1,6-glucan-producing GTF (and/or exogenously produced dextran) and at least one alpha-1,3-glucan-producing GTF in step (b) of producing a food product/precursor allows for production of a dextran-alpha-1,3-glucan graft copolymer as presently disclosed, possibly along with production of dextran and/or alpha-1,3-glucan homopolymer(s) (i.e., alpha-1,6-glucan and/or alpha-1,3-glucan produced independent from the production of graft copolymer). However, it is believed possible that, in some aspects, only dextran and/or alpha-1,3-glucan homopolymer(s) is/are produced with little (e.g., <5 wt % of all glucan products) or no production of graft copolymer.

The molecular weight and/or linkage profile of alpha-glucan produced by a GTF enzyme (dextransucrase or alpha-1,3-glucan-producing GTF) as generally disclosed above can be as observed, for example, in an isolated reaction consisting of, or essentially of, water, sucrose, GTF enzyme and optionally one or more salts and/or buffer. In some aspects, the molecular weight and/or linkage profile of alpha-glucan as produced by one or both of these types of GTF enzyme in a food product/precursor herein may be different from what is produced in the foregoing isolated reaction.

In some aspects, the ratio of a GTF enzyme that synthesizes alpha-1,6-glucan to a GTF enzyme that synthesizes alpha-1,3-glucan in step (b) is about 85:15 to about 95:5. Yet, in some aspects, an alpha-1,6-glucan-producing GTF to alpha-1,3-glucan-producing GTF ratio can be about 97.5:2.5, 95:5, 92.5:7.5, 91:9, 90:10, 89:11, 87.5:12.5, 85:15, 82.5:17.5, 80:20, 70:30, 60:40, 50:50, 40:60, 30:70, 20:80, 10:90, 5:95, or 2.5:97.5, or range between any two of these ratios (e.g., about 82.5:17.5 to 97.5:2.5, 87.5:12.5 to 92.5:7.5, 89:11 to 91:9, 17.5:82.5 to 2.5:97.5, 12.5:87.5 to 7.5:92.5, 11:89 to 9:91). The amount of each enzyme (active enzyme) for purposes of determining a ratio thereof herein can be on a molar, weight, or GTF activity basis, for example. The activity of a GTF enzyme for preparing a ratio herein can optionally be determined as disclosed in U.S. Patent Appl. Publ. No. 2014/0087431, which is incorporated herein by reference, and/or as disclosed in the below Examples. For example, a full (e.g., “100%”) complement of a GTF enzyme for setting up a ratio herein can be that amount of enzyme that can convert most of (e.g., >95%, >98%, >99%), or all of, sucrose in a GTF reaction comprising or consisting of water, sucrose (e.g., 50 or 100 g/L), the GTF, and optionally buffer/salt in a given amount of time (e.g., 6, 12, 18, 24, 30, or 36 hours); such a measured amount can optionally be characterized as a normalized amount of GTF.

A GTF enzyme (or any other enzyme as presently disclosed) for use in a method herein is typically in purified form. A purified enzyme can be essentially free from insoluble and/or soluble components of an organism/cell used to produce the enzyme, and/or any medium that was used for cellular fermentation of the enzyme. In some aspects, a purified enzyme denotes an enzyme preparation that contains less than 10%, 8%, 6%, 5%, 4%, 3%, 2%, 1%, 0.5%, or 0.1% by weight of other material (e.g., polypeptide material) with which the enzyme is natively or recombinantly associated. In some aspects, a GTF and/or any other enzyme herein is not comprised in or otherwise associated with (e.g., expressed by) a microbial (e.g., bacterial, yeast, fungal, algal) cell that might be present (e.g., endogenously or purposely added) in a food product/precursor herein; however, in some aspects a GTF and/or any other enzyme herein is comprised in or otherwise associated with (e.g., expressed by) a microbial (e.g., bacterial, yeast, fungal, algal) cell such as one that heterologously expresses the enzyme(s) (i.e., recombinant cells). Contacting a food product/precursor with a GTF enzyme(s) herein typically is not performed in an oral cavity or other environment in which unpurified/non-isolated GTF enzymes can possibly be present.

A GTF enzyme (or any other enzyme as presently disclosed) for use in a method herein can be comprised in a sterile-filtered preparation, for example. In some aspects, an enzyme can be sterile-filtered inline while applying the enzyme to a food product/precursor during step (b) herein. In some aspects, an enzyme can be added to a food product/precursor that has been pasteurized (after pasteurization), or alternatively an enzyme can be added before pasteurizing the food product/precursor. In some aspects, an enzyme can be added to a food product/precursor that has been fermented (after fermentation), or alternatively an enzyme can be added during or before fermenting the food product/precursor. A GTF enzyme (or any other enzyme as presently disclosed) in some aspects for use in a method herein can be comprised in a preparation (for adding in step [b] herein) that is substantially free of (e.g., <0.5, <0.1, <0.05 wt %) any other enzyme(s) such as a lipase, protease, amylase, mannanase, pectinase, cellulase, and/or p-nitrobenzylesterase; such a preparation typically has little or no detectable activity (ies) of such other enzyme(s).

A food product/precursor herein can be brought into contact with one or more GTF enzymes in step (b) by mixing/stirring/blending, for example. Incubation of GTF enzyme(s) in the food product/precursor can be for a time sufficient, for example, for the GTF(s) to produce alpha-glucan in the food product/precursor, such as for about, or at least about, 0.5, 1, 2, 3, 4, 5, 6, 7, 8, 9, 10, 12, 15, 18, 24, 30, 36, 42, 48, 72, or 96 hours, or for about, or at least about, 5, 10, 15, 20, 25, 30, 35, 40, 45, 50, 55, or 60 days (or a range between any two of these hours and/or days). The temperature for incubating one or more GTF enzymes in a food product/precursor herein can be about 2, 3, 4, 5, 6, 7, 8, 9, 10, 12, 14, 16, 18, 20, 22, 24, 25, 26, 28, 30, 32, 34, 36, 38, 40, 42, 44, 46, 48, 50, 2-5, 2-10, 2-15, 2-20, 2-25, 2-30, 2-35, 2-40, 2-45, 2-50, 3-5, 3-10, 3-15, 3-20, 3-25, 3-30, 3-35, 3-40, 3-45, 3-50, 5-10, 5-15, 5-20, 5-25, 5-30, 5-35, 5-40, 5-45, 5-50, 15-20, 15-25, 15-30, 15-35, 15-40, 15-45, 15-50, 20-25, 20-30, 20-35, 20-40, 20-45, 20-50, 25-30, 25-35, 25-40, 25-45, 25-50, 30-35, 30-40, 30-45, or 30-50° C., for example.

Typically, a food product/precursor brought into contact with a GTF enzyme herein contains water (i.e., it is an aqueous composition), and/or water is introduced to the food product/precursor before or during contacting with GTF enzyme. GTF enzyme can be added to a food product/precursor in dry form (e.g., powder, flakes, lyophilized enzyme preparation) (typically to an aqueous food product/precursor) or wet form. In some aspects, a food product/precursor can be combined with a GTF enzyme under dry conditions (resulting combination is dry), after which time water or an aqueous solution is added, which in turn allows GTF production of alpha-glucan to proceed. The water content of a food product/precursor as provided in step (a), or in step (b) following addition of GTF enzyme, can be about, or at least about, 10, 20, 30, 40, 50, 60, 70, 80, 90, 95, or 99 wt %, for example. The pH of a food product/precursor herein, and/or the pH for incubating one or more GTF enzymes in a food product/precursor herein, can be about 3.0, 3.5, 4.0, 4.5, 5.0, 5.5, 6.0, 6.5, 7.0, 7.5, 8.0, 8.5, 9.0, 9.5, 10.0, 10.5, 4.0-10.0, 4.0-9.0, 4.0-8.0, 4.5-10.0, 4.5-9.0, 4.5-8.0, 5.0-10.0, 5.0-9.0, 5.0-8.0, 5.5-10.0, 5.5-9.0, 5.5-8.0, 6.0-10.0, 6.0-9.0, or 6.0-8.0, for example. A food product/precursor in some aspects can be acidic (e.g., pH≤3.0, 3.2, 3.5, 4.0, 4.5, 5.0, 5.5, 6.0, or 6.5), neutral (e.g., pH 6.5-7.5), or basic/alkaline (e.g., pH>7.5, 8.0, 8.5, 9.0, 9.5).

A GTF enzyme herein can optionally be provided in step (b) of a method by introducing a recombinantly engineered cell (e.g., a microbial cell such as a bacterial or fungal/yeast cell) to the food product/precursor provided in step (a), wherein the cell recombinantly (heterologously) expresses and secretes the GTF enzyme in and/or around the food product/precursor. Such a cell can be that of a microbe that is amenable to recombinant engineering and useful in food processing (e.g., fermentation), such as a microbial cell disclosed herein (as applicable). In some aspects, a recombinantly engineered cell that is provided to the food product/precursor in step (b) can be inactive and/or non-viable in some manner, such as by having been killed (but preferably in a manner that otherwise retains cellular shape/structure). For example, a cell can be rendered inactive and/or non-viable by being irradiated or being treated with a sterilizing agent/chemical (e.g., ethylene oxide). Typically, the means for cell inactivation and/or killing preserves at least some of the three-dimensional shape/structure of the cell, and/or ensures that a GTF enzyme(s) that had been expressed by the cell remains active and typically remains associated with the inactive/non-viable cell (e.g., such as by being associated with a cellular membrane via an optional transmembrane domain or membrane-binding domain of the GTF enzyme [e.g., fused to the GTF]). An inactive/non-viable cell typically is porous, and optionally can be immobilized on a support (e.g., an inert, water-insoluble material, such as of a particle or surface).

In some aspects, a food product/precursor herein can be brought into contact with one or more GTF enzymes by virtue of adding the food product/precursor to an aqueous composition comprising at least sucrose and the one or more GTF enzymes. While the food product/precursor in this aspect has at least some sucrose and water, a food product/precursor in some other aspects does not comprise sucrose and/or water. Such a GTF/sucrose-containing aqueous composition can optionally be referred to herein as a “GTF/sucrose starter composition”. Typically, one or more food precursors as presently disclosed (e.g., ingredients such as a liquid food product/precursor, beverage, RTD, fruit/vegetable purée, syrup, or juice or juice concentrate) can be added to a GTF/sucrose starter composition, although one or more food products themselves as presently disclosed (e.g., fruit or vegetable matter such as pieces [e.g., slices, cubes, or other shaped pieces]) can be added (typically in conjunction with adding a food precursor). In some aspects, a GTF/sucrose starter composition can already comprise at least one food product/precursor, such as any disclosed herein. The initial sucrose concentration of a GTF/sucrose starter composition can be as presently disclosed, for example, such as 5-60%, 5-50%, 5-40%, 10-60%, 10-50%, 10-40%, 20-60%, 20-50%, 20-40%, 30-60%, 30-50%, 30-40%, 40-60%, or 40-50% by weight. In some aspects, a GTF/sucrose starter composition has few (e.g., less than about 1, 0.5, 0.1, 0.05, or 0.01 wt %) or no saccharide compounds (e.g., one or more monosaccharides, disaccharides, oligosaccharides and/or polysaccharides, such as presently disclosed) aside from sucrose. A GTF/sucrose starter composition can comprise at least one alpha-1,6-glucan-producing GTF and/or an alpha-1,3-glucan-producing GTF as presently disclosed, for example. The temperature, pH and/or any other condition/parameter of this methodology (before and/or after adding one or more food products/precursors to a GTF/sucrose starter composition) can be as disclosed herein, for example. In some aspects, a GTF/sucrose starter composition can be incubated for about, at least about, or up to about, 10, 15, 20, 25, 30, 35, 40, 45, 50, 55, 60, 120, 180, 240, 300, 360, 420, 480, 540, 10-45, 10-40, 10-35, 10-30, 10-25, 10-20, 10-15, 15-45, 15-40, 15-35, 15-30, 15-25, 15-20, 20-45, 20-40, 20-35, 20-30, 20-25, 25-45, 25-40, 25-35, or 25-30 minutes, for example, before adding one or more food products/precursors. Any of these foregoing time periods can also apply to the period of time allowed to proceed after adding the food product/precursor to the GTF/sucrose starter composition, until optionally terminating the GTF activity (e.g., heat-inactivation at 90-100° C.) of the final food product/precursor. Typically, the one or more food products/precursors added to the GTF/sucrose starter composition comprises one or more saccharide compounds (e.g., one or more monosaccharides, disaccharides, oligosaccharides and/or polysaccharides, such as presently disclosed). In some aspects, adding one or more food product/precursors to a GTF/sucrose starter composition can be done to control or adjust the degree of thickening and/or texturization desired in the final food product/precursor being produced. The thickening and/or texturization that can be achieved by such methodology can be greater (e.g., about, or at least about, 10%, 25%, 50%, 75%, 100%, 150%, 200%, 250%, 300%, 400%, or 500% greater) than the thickening and/or texturization that would have been achieved if all the ingredients used to make the final food product/precursor had all been combined at about the same time. Merely as examples, the foregoing process of adding one or more food products/precursors to a GTF/sucrose starter composition can be used herein to produce a marmalade, gel/gelatin, pudding, custard, fermented product, or cream, optionally with one or more suspended solid food ingredients such as fruit or vegetable pieces.

A “food product/precursor” (i.e., a food product or precursor) as provided in step (a) of a method in some aspects of the present disclosure method can comprise sucrose that is endogenous to the food product/precursor (e.g., its sucrose is native), and/or can comprise sucrose that has been added to the food product/precursor (either during or after its preparation as an ingredient). Step (a) can thus optionally comprise adding sucrose to the food product/precursor. The sucrose content of a food product/precursor finally provided in step (a) herein, regardless of the original source of the sucrose, can be about, at least about, or less than about, 0.1, 0.5, 1, 2.5, 5, 7.7, 10, 15, 20, 25, 30, 40, 50, 60, or 70 wt %, for example. In some aspects, sucrose can be provided as white refined sucrose, or in an unrefined form such as disclosed in U.S. Pat. No. 9,719,121, for example, which is incorporated herein by reference. Sucrose can optionally be added to a food product/precursor when adding GTF enzyme to the food product/precursor.

In some aspects, a food product/precursor as provided in step (a) of a method herein further comprises at least one disaccharide in addition to sucrose, and/or at least one oligosaccharide. An oligosaccharide can have 3-15 or 3-20 monomeric units (i.e., DP3-DP15 or DP3-DP20), for example (e.g., DP3-DP5, DP3-DP6); thus, in some aspects, a polysaccharide herein has more than 15 or 20 monomeric units. A disaccharide and/or oligosaccharide herein can comprise only glucose monomeric units, for example, and/or one or more other types of monosaccharides (e.g., galactose, fructose, mannose) as monomeric units. Examples of disaccharides herein (in addition to sucrose) include maltose, isomaltose, lactose, lactosucrose, nigerose, leucrose, trehalulose, maltulose, isomaltulose, and turanose. Examples of oligosaccharides herein include gluco-oligosaccharides (gluco-oligomers) such as malto-oligosaccharides (MOS) and isomalto-oligosaccharides (IMO), and galacto-oligosaccharides (GOS). A disaccharide and/or oligosaccharide can be added to a food product/precursor either during or after preparation of the food product/precursor. Such addition can be from a source physically outside of the food product/precursor (i.e., as an ingredient), and/or can be via in situ production in the food/precursor such as by one or more enzymes that are endogenous and/or exogenous to the food/precursor. An enzyme that is added to a food product/precursor (i.e., exogenous enzyme) for producing a disaccharide and/or oligosaccharide can be added, for example, in the same or similar manner in which a GTF enzyme herein is added (e.g., time, temperature, pH), and can be added before, during, or after the addition of GTF enzyme. Such an enzyme can be a transglucosidase (EC [enzyme code] 2.4.1.24) or an amylase, for example. Suitable transglucosidases herein include FoodPro® TGO and those disclosed in U.S. Patent Appl. Publ. Nos. 2008/0229514 or 2015/0240279, or U.S. Pat. No. 4,689,296, all of which are incorporated herein by reference. An EC 2.4.1.24 transglucosidase (also termed as “1,4-alpha-glucan 6-alpha-glucosyltransferase”) can transfer an alpha-D-glucosyl residue of an alpha-1,4-glucan, -oligosaccharide (i.e., MOS), or -disaccharide (i.e., maltose) to the primary hydroxy group of free glucose or glucose in an alpha-1,4-glucan, -oligosaccharide (i.e., MOS), or -disaccharide. Thus, an EC 2.4.1.24 transglucosidase produces isomalto-oligosaccharides (IMO) (e.g., DP3-DP5 or DP3-DP6) in some aspects.

In some aspects, a food product/precursor as provided in step (a) of a method herein has, aside from the sucrose, little (e.g., less than 0.5, 0.25, 0.1, 0.05, 0.025, or 0.01 wt %, or not detectable) or no disaccharides and/or oligosaccharides (or little or no particular disaccharide or oligosaccharide). A food product/precursor as produced in step (b) of a method herein can likewise have, for example, little of no disaccharides and/or oligosaccharides (or little or no particular disaccharide or oligosaccharide), and also have little (e.g., as above) or no sucrose. A disaccharide or oligosaccharide in such aspects can be any as disclosed herein (e.g., lactose, maltose, isomaltose, MOS, IMO, GOS). One or more glycosidase enzymes (glycosidic-active enzyme) can be used, for example, in a food product/precursor to reduce or eliminate the presence of disaccharide(s) and/or oligosaccharide(s), and can be added, for example, in the same or similar manner in which a GTF enzyme herein is added (e.g., time, temperature, pH), and can be added before, during, or after the addition of GTF enzyme. A glycosidase herein can be, for example, an alpha-glucosidase (EC 3.2.1.20). Suitable alpha-glucosidases herein include those disclosed in U.S. Patent Appl. Publ. No. 2015/0240278, which is incorporated herein by reference. In some aspects, an alpha-glucosidase that is used in the disclosed method is able to hydrolyze an alpha-1,4 or alpha-1,6 glucosidic linkage, or and/or is unable to hydrolyze an alpha-1,3 glucosidic linkage. A glycosidase herein can be dosed into a food product/precursor herein at about 0.1-1.5, 0.1-1.25, 0.1-1.0, 0.1-0.75, 0.1-0.5, 0.2-1.5, 0.2-1.25, 0.2-1.0, 0.2-0.75, 0.2-0.5, 0.5-1.5, 0.5-1.25, 0.5-1.0, 0.5-0.75, 0.75-1.5, 0.75-1.25, or 0.75-1.0% (v/w), for example.

A food product/precursor in some aspects can be a flour-based or meal-based dough, baked product (bakery product), or extruded product, such as any of those disclosed in WO2021/034561 or U.S. Patent Appl. Publ. Nos. 2017/0218093 or 2022/0322685, which are incorporated herein by reference. Examples of baked products, or a dough (precursor) thereof, include bread (e.g., buns, sourdough, rye, whole wheat, pita, flatbread, tortilla, cornbread, brioche, white, baguette, bagels, banana, ciabatta, brown, challah, focaccia, multigrain, bread sticks, soda bread, pumpernickel, potato bread, biscuits, English muffins, whole grain, matzo, lavash, croutons, pizza crust) (leavened or unleavened), cake (e.g., carrot cake, red velvet, angle food, pound cake, chocolate, white, black forest, tiramisu, coffee cake, cheesecake, devil's food, upside-down cake, Boston cream pie, Swiss roll, lemon cake, short cake, chiffon cake, butter cake, spice cake, rum cake, sponge cake, marble cake, coconut cake, pandan cake), muffins, brownies, scones, cookies, bars, custards, pies, crackers, pretzels, pastries, pudding and tarts. Examples of an extruded product include pasta (e.g., spaghetti, rotini, fusilli, penne, bucatini, macaroni/maccheroni, rigatoni, fettuccine, linguine, vermicelli, ziti, farfalle, gomiti/elbow, rotelle), cereal (e.g., direct expanded cereal, filled cereal, flakes, breakfast cereal), some bread products (e.g., croutons, bread sticks, flat breads), pre-made cookie-dough, dry and semi-moist pet food (e.g., kibbles), and snacks (e.g., cheese curls, filled pillow puffs, chips [e.g., corn chips, pita chips, processed potato chips, tortilla chips], snack sticks [e.g., vegetable sticks], puffed shaped products such as curls [e.g., cheese curls], balls, tubes, bananas, cups, bowls, disks, baby food puffs). Pasta herein can be extruded (e.g., see above) and/or flattened/rolled (e.g., lasagna), fresh or dried, long or short, minute/soup pasta (pastina), filled (e.g., tortellini, ravioli, agnolotti, tortelli), stretched (e.g., cencioni, corzetti, foglie d'ulivo, orecchiette), and/or egg pasta, for example.

A food product/precursor in some aspects can be a syrup or beverage, for example, such as any of those disclosed in U.S. Patent Appl. Publ. Nos. 2010/0040728, 2017/0006902, 2017/0218093, 2013/0216652, 20180146699, 2009/0123603, 2021/0076724, or 2017/0332670, all of which are incorporated herein by reference. A beverage in some aspects can be a juice (e.g., fruit juice such as orange juice, apple juice, mango juice, peach juice, banana juice, date juice, apricot juice, grapefruit juice, papaya juice, pineapple juice, raspberry juice, strawberry juice, pear juice, tangerine juice, or cherry juice; vegetable juice such as carrot juice, tomato juice, or mixed-vegetable juice), sweetened beverage (soda/soft drink, sweetened tea or coffee), ready-to-drink (RTD), or any other beverage having natural and/or added sugar (sucrose). In some aspects, a beverage is coffee or tea.

In some aspects, a food product/precursor provided in step (a) of a method herein is fermented, while in some aspects a food product/precursor is fermented (or further fermented) during or after performing step (b). Thus, step (a) of a method herein can optionally comprise a step of fermenting a food product/precursor (e.g., before or after adding sucrose, if applicable). Thus, step (b) of a method herein can optionally comprise fermenting the food product/precursor while contacting it with a GTF enzyme. Thus, a method herein can optionally comprise, following step (b), a step of fermenting the food product/precursor. One or more bacterial and/or yeast cultures can be used for fermentation of a food product/precursor herein. Suitable bacteria for food fermentation herein include lactic acid bacteria, for example, such as Lactobacillus family species such as those of the Pediococcus genus (e.g., P. acidilactici, P. pentosaceus), Lactobacillus genus (e.g., L. sakei, L. fermentum [formerly L. cellobiosus], L. rhamnosus, L. plantarum, L. brevus, L. kefir, L. casei, L. paracasei, L. acidophilus, L. salivarius, L. buchneri, L. helveticus, L. reuteri, L. johnsonii, L. crispatus, L. gasseri, L. delbruecki such as subsp. L. bulgaricus), Lactococcus genus (e.g., L. lactis such as subsp. L. cremoris), Leuconostoc genus (e.g., L. citreum, L. mesenteroides), and Streptococcus genus (e.g., S. thermophilus). Suitable bacteria for food fermentation in some aspects can be species from the Bifidobacterium genus (e.g., B. bifidum, B. lactis, B. longum, B. animalis, B. breve, B. infantis), Komagataeibacter ssp., Liquorilactobacillus ssp. (e.g., Liquorilactobacillus neglii, Liquorilactobacillus ghanensis), Gluconobacter ssp. or Propionibacterium genus (e.g., P. freudenreichii such as subsp. P. shermanii) (propionic acid bacteria). In some aspects, a bacteria for food fermentation herein can be characterized as Gram-positive, sphere-shaped, rod-shaped, anaerobic, aerobic, acid-tolerant, non-sporulating, GRAS (generally regarded as safe), and/or probiotic. A bacteria for food fermentation (e.g., plant-based fresh fermented product) in some aspects can be an acidic culture/strain (or mix) (e.g., (Danisco® VEGE 053, Danisco® VEGE 011, available from IFF) that produces food with a pH of about, for example, 2.5-4.5, 3.0-4.5, 4.2-4.4, or 4.3, or it can be a mild culture/strain (or mix) (e.g., Danisco® VEGE 022, Danisco® VEGE 053, Danisco® VEGE 022, Danisco® VEGE 047, Danisco® VEGE 061, available from IFF) that produces food with a pH of about, for example, 4.6-5.5, 4.6-6.0, 4.5-4.7, or 4.6. A bacteria herein (e.g., mild of acidic) can optionally be mesophilic (temperature for optimal growth typically at 20 to 25° C. [room temperature]). A mix of bacteria in a culture for food fermentation can comprise one, two, three, four, five, six or more different species and/or sub-species of bacteria, for example. Suitable yeast for food fermentation in some aspects include species from the Saccharomyces genus (e.g., S. cerevisiae, S. pastorianus, S. boulardii, S. kluyveri, S. vitulinus), Pediococcus ssp. (e.g., Pediococcus pentosaceus), K. lactis, Dekkera ssp. (e.g., Dekkera bruxellensis), Zygosaccharomyces ssp. (e.g., Zygosaccharomyces bailii), Brettanomyces ssp., Pichia genus (e.g., P. kluyveri, P. fermentans), Geotrichum, Debaryomyces and Candida genus (e.g., C. humilis, C. famata). A yeast in some aspects can be characterized as baker's (baking) yeast, brewing yeast, wine-making yeast, probiotic yeast, budding/fission yeast, or GRAS. A mix of yeast in a culture for food fermentation can comprise one, two, three, four, five, six or more different species and/or sub-species of yeast, for example. A food product/precursor that is fermented or will be fermented can be a plant-based fresh fermented product herein, beer, beer wort, wine, pomace, cider, miso, kimchi, sauerkraut, pickles/pickle juice, soybean curd, tofu, kombucha, soy sauce, bread, sourdough, or meat, for example.

A food product/precursor in some aspects can be a confectionary, for instance. Examples of confectioneries herein include boiled sugars (hard boiled candies [i.e., hard candy]), dragees, jelly candies, gums, licorice, chews, caramels, toffee, fudge, chewing gums, bubble gums, nougat, chewy pastes, halawa, tablets, lozenges, icing, frosting, pudding, gels (e.g., fruit gels, gelatin dessert), aerated confectioneries, marshmallows, baked confectioneries.

A food product/precursor in some aspects can be a non-dairy food product/precursor. For example, a non-dairy food product/precursor can be a plant-based milk (milk substitute) or comprise a plant-based milk (and lack, or have little of [e.g., <0.5 wt %], any dairy ingredient[s] such as lactose, whey, casein, and/or milk fat). In some aspects, a non-dairy food product/precursor is fermented (e.g., a non-dairy yogurt product/precursor such as a plant-based yogurt product/precursor). Plant-based ingredient(s) forming the basis for a non-dairy food product/precursor herein can be from nuts/seeds (e.g., almonds, cashews, macadamias, hemp seed, quinoa, flax seed), grains/cereal (e.g., oats, rice), fruit (e.g., coconut, banana), or vegetables (e.g., legumes such as beans [e.g., soybeans, mug beans] and peas), for example. In some aspects, a non-dairy food product/precursor is a milk of any of the foregoing nuts/seeds, grains/cereal, fruit, or vegetables; a fermented form of any of these milks can be a yogurt, for example. Yet, in some aspects, a food product/precursor can comprise any of the foregoing plant-based ingredient(s) and be in any suitable food/precursor form disclosed herein (i.e., the food product/precursor need not be characterized as a non-dairy food product/precursor such as plant-based milk).

A food product/precursor in some aspects can be a cream soup, gravy, sauce (e.g., tomato sauce), salad dressing, mayonnaise, jam, jelly, marmalade, syrup, pie filling, batter for fried foods, batter for pancakes/waffles, cake icing and glazes, whipped topping, pet food, or animal/livestock feed.

A food product/precursor in some aspects can comprise one or more additional ingredients such as a vegetable component (e.g., vegetable oil, vegetable protein, vegetable carbohydrates), enzyme, fat, oil, flavoring agent, microbial culture (e.g., probiotic culture), salt, sweetener, acid, fruit/vegetable (e.g., orange, apple, mango, peach, plum, banana, date, apricot, grapefruit, papaya, pineapple, raspberry, strawberry, blueberry, blackberry, pear, tangerine, cherry, grape, raisin, currant, melon, watermelon, cantaloupe, honeydew melon, kiwi, lemon, lime, carrot, tomato), or fruit/vegetable juice (juice concentrate), purée, or other processed form (e.g., sliced, cubed, chopped pieces) of a fruit/vegetable as disclosed, or any other component suitable for use as an ingredient in a food product/precursor. Such one or more additional ingredients can be as disclosed in U.S. Patent Appl. Publ. Nos. 2016/0122445 or 2017/0218093 (both incorporated herein by reference), for example, and/or can be natural or artificial. Examples of ingredients suitable as sweeteners (or for any other purpose such as flavoring) include acesulfame potassium, advantame, agave syrup, alitame, aspartame, barley malt syrup, birch syrup, brazzein, brown rice syrup, cane juice, caramel, coconut palm sugar, corn syrup, curculin, cyclamate, dextrose, erythritol, fructo-oligosaccharide, fructose (levulose), galactose, glucose (dextrose), glycerol (glycerin), glycyrrhizin, golden syrup, high fructose corn syrup (e.g., HFCS-42, -55, -90), high maltose corn syrup (HMCS), honey, hydrogenated starch hydrolysate (HSH), isomalto-oligosaccharide (IMO), inulin, inverted sugar, isomalt, lactitol, lactose, maltitol, maltodextrin, maltose, mannitol, maple syrup, miraculin, molasses (e.g., blackstrap molasses), monatin, monellin, monk fruit, neohesperidin dihydrochalcone, neotame, palm sugar, pentadin, polydextrose, rapadura, refiners syrup, saccharin, sorbitol (glucitol), sorghum syrup, stevia/steviol glycoside (e.g., a rebaudioside such as rebaudioside A, rebaudioside D, or rebaudioside M), sucralose, sugar alcohol, tagatose, thaumatin, trehalose, xylitol, and yacon syrup.

In some aspects, a food product/precursor comprises at least one isoflavone glycoside herein. Examples of such a food product/precursor include legumes (e.g., soybeans, beans, green beans, pinto beans, black beans, peas [green or yellow], chickpeas, fava beans, pistachios, peanuts), currants, raisins, coffee beans, and coffee, or any product/derivative thereof (e.g., powder, flour, meal, yogurt, tofu, miso, natto, tempeh, or as disclosed herein).

In some aspects, a food product/precursor as produced by a method of the present disclosure can be concentrated, dried (e.g., to a powder), reconstituted (following concentration or drying), or processed (e.g., frozen) in any other manner. Examples of such products include sweetened milk, concentrated milk, condensed milk (e.g., sweetened condensed milk), evaporated milk, dried milk powder, frozen dairy product (e.g., ice cream) concentrated juice, or dried juice powder.

In some aspects, a method herein can further comprise a step of freezing a food product/precursor (e.g., a dairy food product/precursor, or a plant-based food product/precursor) after step (b). This method can produce a plant-based ice cream or frozen yogurt, for example. Freezing can be done at about −10, −15, −20, −25, −30, −35, −40, −20 to −40, −25 to −35° C., for example. A frozen product in some aspects—in which method step (b) comprises using at least an alpha-1,3-glucan-producing GTF herein—can have an improved melting profile (e.g., slower melting) as compared to a suitable control (e.g., a frozen product that was not treated with an alpha-1,3-glucan-producing GTF, but otherwise made with the same ingredients and process steps). Slower melting of a frozen product in some aspects can be melting that is reduced by about, or at least about, 10%, 20%, 30%, 40%, 50%, 60%, 70%, 80%, or 90% as compared to the melting of a suitable control. Melting can refer to that melting which occurs within about 45, 60, 75, 90, 100, 120, 150, or 60-120 minutes after placing a frozen product (from freezing conditions) into an ambient temperature (e.g., about 20, 25, 18-25, or 20-25° C.) or an elevated temperature (e.g., about 25-38, 25-35, 25-32, or 25-30° C.), for example. Melting can optionally be as measured according to the below Examples or as disclosed in Granger et al. (2005, Int. Dairy J. 15 (3): 255-262, incorporated herein by reference), for instance.

A food product/precursor in some aspects after step (b) of a method herein has reduced off-flavor as compared to the food product/precursor as it existed before step (b) (i.e. as it existed before being treated with one or more GTF enzymes). Off-flavor can be reduced by about, or at least about 25%, 50%, 70%, 75%, 80%, 85%, 90%, or 95%, for example. Such a reduction in off-flavor can, in some aspects, be as compared to (i) the off-flavor that existed before the contacting with GTF(s), or (ii) the off-flavor of a suitable control (e.g., only difference being no GTF treatment). Off-flavor herein can comprise bitterness and/or astringency, for example. Bitterness can optionally be measured in International Bitterness Units (IBU). Off-flavor can be as measured according to the below Examples, for instance, such as by sensory evaluation.

A food product/precursor in some aspects after step (b) of a method herein has a reduced content of at least one isoflavone glycoside and/or an increased content of at least one glucosylated isoflavone glycoside, as compared to the food product/precursor before step (b). The reduction in the content of one, or a combination of, isoflavone glycoside(s) herein can be by about, or at least about, 10%, 15%, 20%, 25%, 30%, 40%, 50%, 60%, 65%, 20-65%, 20-60%, 25-65%, or 25-60% by weight, for example, as compared to the isoflavone glycoside(s) content of the food product/precursor as it existed before step (b). The increase in the content of one, or a combination of, glucosylated isoflavone glycoside(s) herein can be by about, or at least about, 10%, 25%, 50%, 100%, 250%, 500%, or 1000% by weight, for example, as compared to the glucosylated isoflavone glycoside(s) content of the food product/precursor as it existed before step (b).

A food product/precursor in some aspects after step (b) of a method herein optionally has one or more of the following features as compared to the food product/precursor as it existed before step (b) (i.e. as it existed before being treated with one or more GTF enzymes):
(I) reduced sugar content,
(II) increased texture, such texture optionally comprising increased thickness and/or increased mouthfeel,
(III) improved physical appearance,
(IV) reduced calories,
(V) increased dietary fiber, and/or
(VI) increased stringiness or stretchability.

In some aspects, the sugar content (e.g., wt %) in a food product/precursor after step (b) can be reduced by about, or at least about, 10%, 15%, 20%, 25%, 30%, 35%, 40%, 45%, 50%, 55%, 60%, 65%, 20-65%, 20-60%, 25-65%, or 25-60%, as compared to the sugar content of the food product/precursor as it existed before step (b). In some aspects, this reduction is with respect to all sugars in the food product/precursor, whereas in other aspects this reduction is with respect to a particular sugar such a sucrose. Sugar can be any as presently disclosed, for example. Sugar content herein can be measured by HPLC, for example, such as disclosed in the below Examples.

In some aspects, the texture of a food product/precursor after step (b) can be increased by about, or at least about, 25%, 50%, 75%, 100%, 200%, 300%, 400%, 500%, 750%, 1000%, 1250%, 1500%, 1750%, 2000%, 2250%, 2500%, 3000%, 3500%, 4000%, 4500%, 5000%, 5500%, 6000%, 6500%, or 7000% as compared to the texture of the food product/precursor as it existed before step (b). Texture can be in terms of thickness or mouthfeel, and/or measured in units of Pascal-seconds (Pa·s) or cP, for example, any of which can optionally be measured according to the below Examples. In some aspects, texture (thickness) can be measured by determining food product/precursor viscosity when extracted at a shear rate of about 11 to 12 Hz (e.g., 11.7 Hz). Texture (mouthfeel) can be measured by determining food product/precursor viscosity when extracted at a shear rate of about 248-250 Hz (e.g., 249 Hz), for example.

In some aspects, a food product/precursor after step (b) has an improved physical appearance as compared to the physical appearance of the food product/precursor as it existed before step (b). Improved physical appearance can be increased homogeneity (e.g., visual homogeneity) and/or increased shininess (e.g., visual shininess), for example; such increase(s) can be by about, or at least about, 5%, 10%, 20%, 25%, 30%, 40%, or 50% in some aspects.

In some aspects, the dietary caloric content (calories that can be accessed during digestion) of a food product/precursor after step (b) can be decreased by about, or at least about, 5%, 10%, 20%, 25%, 30%, 40%, 50%, 60%, 70%, or 75% as compared to the dietary caloric content of the food product/precursor as it existed before step (b).

In some aspects, the dietary fiber content (e.g., weight percent) of a food product/precursor after step (b) can be increased by about, or at least about, 5%, 10%, 25%, 50%, 75%, 100%, 200%, 300%, 400%, or 500% as compared to the dietary fiber content of the food product/precursor as it existed before step (b).

In some aspects, such as when contacting a food product/precursor comprising maltose (and/or IMO) (in addition to sucrose) with an alpha-1,3-glucan-producing GTF and/or an alpha-1,6-glucan-producing GTF herein, a high reduction (e.g., at least about 35%, 40%, 45%, 50%, 55%, or 60%) in sugar can be realized without significantly changing the texture (e.g., change less than 10%, 9%, 8%, 7%, 6%, 5%, 4%, 3%, 2%, 1%; e.g., thickness or mouthfeel) of the food product/precursor.

Some aspects of the present disclosure concern a food product/precursor as produced by a GTF treatment method herein. Examples of such products/precursors are any food product/precursor as disclosed herein. Typically, such a food product/precursor can have any feature as disclosed herein (e.g., reduced sugar content, increased texture, increased stringiness (stretchability), improved physical appearance, reduced caloric content, increased dietary fiber, pH, temperature, age, hardness, reduced melt rate), as appropriate/applicable. Typically, such a food product/precursor comprises at least one GTF enzyme as presently disclosed, and/or an alpha-glucan as presently disclosed.

In some aspects, an isoflavone glycoside herein serves as an acceptor/primer for in situ alpha-glucan synthesis by a GTF enzyme in the food/precursor. Thus, some aspects of the present disclosure concern a polysaccharide or alpha-glucan molecule comprising at least:
(i) alpha-1,6-glucan or alpha-1,3-glucan as disclosed herein, and
(ii) an isoflavone glycoside (e.g., genistin or daidzin).

Some aspects of the present disclosure concern a method of glucosylating an isoflavone glycoside, which method can optionally be characterized as an isoflavone glycoside glucosylation method. Such a method can comprise a step of providing a composition that comprises at least water, sucrose, an isoflavone glycoside, and a glucosyltransferase enzyme herein, and wherein the glucosyltransferase enzyme is selected from:
(i) a glucosyltransferase enzyme that synthesizes alpha-1,6-glucan, wherein at least about 50% of the glycosidic linkages of the alpha-1,6-glucan are alpha-1,6 linkages, and/or
(ii) a glucosyltransferase enzyme that synthesizes alpha-1,3-glucan, wherein at least about 50% of the glycosidic linkages of the alpha-1,3-glucan are alpha-1,3 linkages,
wherein at least one glucosylated form of the isoflavone glycoside (glucosylated isoflavone glycoside) is produced in the composition. The providing step of such a method can optionally be characterized as a step of contacting an isoflavone glycoside with a glucosyltransferase enzyme in the presence of at least water and sucrose. Typically, an isoflavone glycoside glucosylation method also produces at least one alpha-glucan as presently disclosed, such as an alpha-1,6-glucan, an alpha-1,3-glucan, and/or a graft copolymer herein.

An alpha-1,6-glucan-synthesizing glucosyltransferase enzyme in an isoflavone glycoside glucosylation method can be any as presently disclosed herein. An alpha-1,3-glucan-synthesizing glucosyltransferase enzyme in an isoflavone glycoside glucosylation method can be any as presently disclosed herein. Any condition and/or parameter for using any of these enzymes in this method can be as presently disclosed herein (e.g., temperature, time, water content, sucrose content, GTF enzyme content).

A glucosylated isoflavone glycoside herein (e.g., product [s] of an isoflavone glycoside glucosylation method) is believed to comprise 1, 2, 3, 4, 5, 6, 7, 8, 9, 10, or more glucose monomeric units (typically in a chain) added to the original isoflavone glycoside, for example. An isoflavone glycoside can optionally be characterized herein to serve as an acceptor/primer for alpha-glucan synthesis by a GTF enzyme in a composition of the disclosure. Thus, some aspects of the present disclosure concern a polysaccharide or alpha-glucan molecule comprising at least:
(i) alpha-1,6-glucan or alpha-1,3-glucan as disclosed herein, and
(ii) a isoflavone glycoside herein (e.g. daidzin or genistin);
wherein portion (i) is in glycosidic linkage with portion (ii), and portion (ii) is at the reducing end of the alpha-glucan molecule (e.g., by virtue of having used the isoflavone glycoside of (ii) as an acceptor for priming synthesis of the alpha-1,6-glucan or alpha-1,3-glucan). The DP or DPw of the alpha-glucan can be any DP or DPw value disclosed herein, for example.

In some aspects, a composition in which a glucosylated isoflavone glycoside can be produced in an isoflavone glycoside glucosylation method can be any as presently disclosed herein, such as a food precursor/product. Yet, in some aspects, such a composition can be in the form of, and/or comprised in, a household care product, personal care product, industrial product, ingestible product (e.g., food product/precursor such as any disclosed herein), or pharmaceutical product, for example, such as described in any of U.S. Patent Appl. Publ. Nos. 2018/0022834, 2018/0237816, 2018/0230241, 20180079832, 2016/0311935, 2016/0304629, 2015/0232785, 2015/0368594, 2015/0368595, 2016/0122445, 2019/0202942, or 2019/0309096, or International Patent Appl. Publ. No. WO2016/133734, which are all incorporated herein by reference. In some aspects, a composition can comprise at least one component/ingredient of a household care product, personal care product, industrial product, pharmaceutical product, or ingestible product (e.g., food product) as disclosed in any of the foregoing publications and/or as presently disclosed. A glucosylated isoflavone glycoside can be produced in situ in a composition, and/or can be produced in a separate or isolated GTF reaction composition and then introduced as an ingredient for making a composition.

A glucosylated isoflavone glycoside in some aspects can provide stability to an emulsification or dispersion. Thus, a glucosylated isoflavone glycoside can optionally be characterized as an emulsification aid or dispersion aid. An emulsification or dispersion stabilized by a glucosylated isoflavone glycoside herein can be that of any household care product, personal care product, industrial product, ingestible product (e.g., food product/precursor such as any disclosed herein), or pharmaceutical product, for example. The “stability” (or the quality of being “stable”) of a dispersion or emulsion herein is, for example, the ability of dispersed particles of a dispersion, or liquid droplets dispersed in another liquid (emulsion), to remain dispersed (e.g., about, or at least about, 70, 75, 80, 85, 90, 95, 96, 97, 98, 99, or 100 wt % of the particles of the dispersion or liquid droplets of the emulsion are in a dispersed state) for a period of about, or at least about, 2, 4, 6, 9, 12, 18, 24, 30, or 36 months following initial preparation of the dispersion or emulsion. A stable dispersion or emulsion in some aspects can resist total sedimentation, flocculation, and/or coalescence of dispersed/emulsified material.

In some aspects, such as with an aqueous dispersion or aqueous emulsification stabilized by a glucosylated isoflavone glycoside herein, particles or liquid (e.g., oil) droplets thereof are dispersed through about, or at least about, 30%, 40%, 50%, 60%, 70%, 80%, 85%, 90%, 95%, 98%, 99%, or 100% of the volume of the aqueous composition. In some aspects, such a level of dispersion or emulsification is contemplated to be for a time (typically beginning from initial preparation of the dispersion or emulsification) of about, at least about, or up to about, 0.5, 1, 2, 4, 6, 8, 10, 20, 30, 60, 90, 120, 150, 180, 210, 240, 270, 300, 330, or 360 days, or 1, 2, or 3 years. Based on the foregoing dispersion and/or emulsion stability features of an aqueous composition, it is contemplated that an aqueous composition is suitable for use, for example, in an application/product in which dispersion or emulsion stabilization improves the performance of the application/product. Examples of such applications/products can be as disclosed herein, such as milk/dairy products (e.g., yogurt, ice cream, cream), mayonnaise, salad dressings, beverages/tonics as carriers for delivering non-polar bioactive ingredients, cosmetic or pharmaceutical lotions/creams/foams/serums, or pharmaceutical carrier or encapsulation systems.

Some aspects of an isoflavone glycoside glucosylation method comprise fermenting a food product/precursor after the step of providing a food product/precursor that comprises at least water, sucrose, an isoflavone glycoside, and a glucosyltransferase enzyme. Thus, in such a method, a process for fermenting the food product/precursor is only commenced after some of (e.g., about, or at least about, 20, 30, 40, 50, 60, 70, 80, 90, 95, 98, 99, or 99.5 percent by weight of), or all of, the isoflavone glucoside that was initially present in the food product/precursor has been glucosylated by the one or more glucosyltransferase enzymes.

Non-limiting examples of compositions and methods disclosed herein include:
1. A method of producing a food product/precursor, the method comprising: (a) providing a food product/precursor that comprises at least water, sucrose and a plant-based material (e.g., a legume food product/precursor), and (b) contacting the food product/precursor with at least: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, typically wherein at least one alpha-glucan is produced in the food product/precursor, whereby the food product/food precursor, after step (b), has reduced off-flavor as compared to the food product/precursor before step (b), and optionally has a reduced sugar content as compared to the food product/precursor before step (b).
2. The method of embodiment 1, wherein the food product/food precursor, after step (b), has a reduced content of at least one isoflavone glycoside and/or an increased content of at least one glucosylated isoflavone glycoside, as compared to the food product/precursor before step (b), optionally wherein the reduced off-flavor results from the reduced content of at least one isoflavone glycoside.
3. The method of embodiment 2, wherein the at least one isoflavone glycoside is daidzin or genistin.
4. The method of embodiment 1, 2, or 3, wherein the off-flavor comprises bitterness and/or astringency.
5. The method of embodiment 1, 2, 3, or 4, wherein: the glucosyltransferase enzyme that synthesizes alpha-1,6-glucan comprises an amino acid sequence that is at least 90% identical to SEQ ID NO:1, 2, 11, 12, 14, 15, 16, 17, or 18, and/or the glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO:7, residues 55-960 of SEQ ID NO: 8, residues 55-960 of SEQ ID NO:9, or SEQ ID NO:13.
6. The method of embodiment 1, 2, 3, 4, or 5, wherein step (a) comprises adding sucrose to the food product/precursor.
7. The method of embodiment 1, 2, 3, 4, 5, or 6, wherein the food product/precursor of step (a) is fermented, or the method further comprises, during or after step (b), fermenting the food product/precursor.
8. The method of embodiment 1, 2, 3, 4, 5, 6, or 7, wherein the food product/precursor is a non-dairy food product/precursor (e.g., a plant-based yogurt product/precursor), optionally wherein the food product/precursor produced by the method is a fresh fermented product/precursor (e.g., a plant-based yogurt product/precursor).
9. A food product/precursor produced by the method of embodiment 1, 2, 3, 4, 5, 6, 7, 8, or 19.
10. A method of glucosylating an isoflavone glycoside, the method comprising: providing a composition that comprises at least water, sucrose, an isoflavone glycoside, and a glucosyltransferase enzyme (or contacting an isoflavone glycoside with a glucosyltransferase enzyme in the presence of at least water and sucrose), wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages, and/or (ii) a glucosyltransferase enzyme that synthesizes alpha-1,3-glucan, wherein at least about 50% of the glycosidic linkages of the alpha-1,3-glucan are alpha-1,3 linkages, wherein at least one glucosylated form of the isoflavone glycoside (glucosylated isoflavone glycoside) is produced in the composition, and typically wherein at least one alpha-glucan (e.g., the alpha-1,6-glucan, the alpha-1,3-glucan, and/or a graft copolymer herein) is produced in the composition.
11. The method of embodiment 10, wherein the isoflavone glycoside is daidzin or genistin.
12. The method of embodiment 10 or 11, wherein: the glucosyltransferase enzyme that synthesizes alpha-1,6-glucan comprises an amino acid sequence that is at least 90% identical to SEQ ID NO:1, 2, 11, 12, 14, 15, 16, 17, or 18, and/or the glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO:7, residues 55-960 of SEQ ID NO: 8, residues 55-960 of SEQ ID NO:9, or SEQ ID NO:13.
13. The method of embodiment 10, 11, or 12, wherein the composition is a food product/precursor.
14. The method of embodiment 13, wherein the food product/precursor is a plant-based food product/precursor (e.g., a legume food product/precursor), optionally wherein the food product/precursor is a fresh fermented product/precursor (e.g., a plant-based yogurt product/precursor).
15. The method of embodiment 13 or 14, further comprising: fermenting the food product/precursor after the providing step.
16. A composition, or a glucosylated isoflavone glycoside, produced by the method of embodiment 10, 11, 12, 13, 14, or 15.
17. A composition comprising a glucosylated isoflavone glycoside, wherein the glucosylated isoflavone glycoside is produced by contacting an isoflavone glycoside with a glucosyltransferase enzyme in the presence of at least water and sucrose, wherein the glucosyltransferase enzyme is selected from: (i) a glucosyltransferase enzyme that synthesizes alpha-1,6-glucan, wherein at least about 50% of the glycosidic linkages of the alpha-1,6-glucan are alpha-1,6 linkages (e.g., as recited in embodiment 5 or 12), and/or (ii) a glucosyltransferase enzyme that synthesizes alpha-1,3-glucan, wherein at least about 50% of the glycosidic linkages of the alpha-1,3-glucan are alpha-1,3 linkages (e.g., as recited in embodiment 5 or 12), optionally wherein the composition is a food product/precursor, and/or optionally wherein the glucosylated isoflavone glycoside is produced in situ in the composition or is produced in an isolated GTF reaction and then added as an ingredient for producing the composition.
18. The composition of embodiment 17, wherein the isoflavone glycoside is daidzin or genistin.
19. A method according to embodiment 1, 2, 3, 4, 5, 6, 7, 8, 10, 11, 12, 13, 14, or 15, but wherein the glucosyltransferase enzyme is a modified (non-native) glucosyltransferase enzyme that synthesizes alpha-1,3-glucan, wherein at least about 50% of the glycosidic linkages of the alpha-1,3-glucan are alpha-1,3 linkages, wherein the modified glucosyltransferase enzyme comprises one or more amino acid substitution(s) at a position(s) corresponding with amino acid residue(s) Tyr-185, Val-186, Leu-513, Gln-588, Phe-607, Ile-608, Lys-625, Arg-741, Val-1188, Lys-1327, Glu-1332, Asp-1418, Ala-1419, Ser-1420, Thr-1421, Arg-1424, Leu-1425, Thr-1431, and/or Glu-1450 of SEQ ID NO:10, and optionally wherein the modified glucosyltransferase comprises an amino acid sequence that is at least about 90% identical to SEQ ID NO:5, 6, 7, 8, or 9, or amino acid residues 55-960 of SEQ ID NO:5, amino acid residues 54-957 of SEQ ID NO:6, amino acid residues 55-960 of SEQ ID NO:7, amino acid residues 55-960 of SEQ ID NO:8, or amino acid residues 55-960 of SEQ ID NO:9, optionally wherein the modified glucosyltransferase has enhanced performance and/or stability benefit(s), and optionally wherein the method further includes using a glucosyltransferase enzyme that synthesizes alpha-1,6-glucan, wherein at least about 50% of the glycosidic linkages of the alpha-1,6-glucan are alpha-1,6 linkages.

### EXAMPLES

The present disclosure is further exemplified in the following Examples. It should be understood that these Examples, while indicating certain aspects herein, are given by way of illustration only. From the above discussion and these Examples, one skilled in the art can ascertain the essential characteristics of the disclosed embodiments, and without departing from the spirit and scope thereof, can make various changes and modifications to adapt the disclosed embodiments to various uses and conditions.

### Materials/Methods

### HPLC Sugar Content Analysis

Sugar composition was measured by high performance liquid chromatography (HPLC) with a Waters® 2695 Serrations module or a ThermoScientific Dionex™ UltiMate 3000 HPLC, equipped with a Phenomonex Rezex™ RPM-Monosaccharide Pb²⁺ column (300 mm×7.8 mm), and an RI-detector. Water was used as the mobile phase at a flow rate of 0.400 mL/min. The column temperature was 70° C. Samples were prepared for HPLC injection by an appropriate dilution in water, optionally centrifugation (10 min at 15,000 rpm), and a sterile filtration. Signals from the HPLC were quantified against calibration standards of sugars eluting at the same time. Sugar reduction was calculated by subtracting the total sum of mono- and di-saccharides in the test sample from the total sum of mono- and di-saccharides in the reference sample without enzymes added.

### Yogurt Preparation

Pre-pasteurized (72° C. for 15 s) bulk-blended skimmed milk (0.1% fat) (Arla Foods, Denmark) stored at 4-6° C. was standardized to a desired protein (% w/w), fat (% w/w) and sucrose (% w/w) content by addition of skimmed milk powder (33% protein, 1.2% fat, 54% carbohydrate) from BBA Lactalis (Laval, Mayenne, France), cream (38% fat) from Arla Foods, and sucrose (Granulated Sugar 500, Nordic Sugar A/S, Denmark). The thus prepared standardized milk was then pasteurized and homogenized in a plate heat exchange pasteurizer. Homogenization was performed at 65° C. at 200 bar and pasteurization at 95° C. for 6 minutes, and then the milk was cooled to 43° C. The milk was inoculated with a thermophilic starter culture at an inoculation rate of 20 DCU/100 L; all cultures were from IFF. Fermentation was conducted until pH 4.60 after which the product was cooled to 24° C. The resulting yogurts were stored at 4-6° C. for viscosity measurements.

### Method for Measuring Apparent Viscosity

A rotational rheological test was employed to evaluate the viscosity of the produced samples. Flow curves were obtained with an Anton Paar MCR302 rheometer (Anton Paar GmbH, Ostfildern, Germany) using an ST22-4V-40 vane geometry for alu cups. Samples were filled into C-CC27 alu cups and stored at 5° C. for at least 5 hours before analysis. The shear rate intervals applied to the samples were 0.1-350 s⁻¹, which defines the up-curve, and the reverse operation explains the down-curve (350-0.1 s⁻¹). The value of the measuring point duration was selected to be at least as long as the value of the reciprocal shear rate, which is valid for the up-curve. The tests were performed under a constant temperature of 10° C., and each sample was analyzed in duplicate. A water bath was connected to the rheometer to ensure isothermal conditions.

The apparent viscosity was assessed from the flow curves, which is appropriate for fluids where the ratio of shear stress to shear rate varies with the shear rate. The apparent viscosity was extracted at either shear rate 11.7 Hz or 249 Hz. The apparent viscosity extracted at shear rate 11.7 Hz indicated the “thickness” of the sample. The apparent viscosity extracted at shear rate 249 s⁻¹ (249 Hz) was correlated to the sensory perception of “mouthfeel”.

### LC-MS

### Chromatographic Conditions:

The chromatographic system was a UPLC system running at 400 μl/min. Solvent A was water/formic acid (1000/1) and solvent B was acetonitrile/formic acid (1000/1). The gradient started at 1% B (0 min) and ended at 25% B at 34 min followed by a rinsing step to 95% B and an equilibration step back to 1% B (5 min). The column was a reversed phase UPLC C18 column (100×2.1 mm id.).

### Mass Spectrometric Conditions:

The mass spectrometer was a high-resolution orbitrap-type instrument run in positive mode. The interface was electrospray. The MS scans were recorded at 120 k resolution in the m/z range 150-1500. The MS2 scans were recorded at 15 k resolution in data-dependent mode with a cycle time of 1.5 sec. The activation type was HCD.

### Data Processing:

The data were processed using Genedata Expressionist®. All identified features were searched against a library of masses representing a range of isoflavones and their glucosylated isoforms, including the core structures daidzein, genistein, glycitein, daidzin, genistin, glycitin and glucosylated forms having from 2-10 hexoses.

### Example 1

### In Situ Glucosylation of Isoflavone Glycosides in a Fresh-Fermented Preparation for Off-Flavor Reduction

The sugar reduction and flavor effects of GTF 0768 (SEQ ID NO:1) and vGTFJ (SEQ ID NO:3), alone or in combination, were investigated in a plant-based fresh-fermented product. For the production of each fresh-fermented soy product, a soy base was made by mixing 276 g of a commercial soy drink (Naturli' Økologisk Soya Drik Usødet, which includes 2.1% fat, 3.7% protein and 0.6% carbohydrate; Naturli' Foods A/S, Vejen, Denmark) and 24 g of sucrose (equaled 8 wt % sucrose in the final base) for 10 minutes at 50° C. Subsequently, the base was pasteurized at 95° C. for 3 minutes. After the heat-treatment, each base was cooled to 43° C. (fermentation temperature). All the samples were then fermented with Danisco® VEGE033 culture (20 DCU/100 mL). Three sample variants were produced. Sample 1 acted as a reference and included no added GTF enzymes. For Sample 2, GTF 0768 and vGTFJ were added together with the culture at the normalized ratio of 75% and 25% respectively. For Sample 3, only vGTFJ was added with the culture inoculation. The individual dosages of the GTF 0768 and/or vGTFJ enzymes were normalized in which 100% dosage was the dosage necessary to provide full sucrose conversion (by GTF 0768 or vGTFJ alone) when added at the inoculation step. All the samples were fermented until a pH of 4.8 was reached; the time to reach this pH was within 7.5-8.5 hours for all the samples. The fermentations were stopped by shearing with a hand mixer (IdeenWelt, Burgwedel, Germany) for 20 seconds at the lowest speed setting and then immediately cooling the samples to 5° C.

The sugar content (by HPLC) and texture of each yogurt sample was assessed according to the Materials/Methods. This work confirmed the conversion of sucrose into polysaccharide components by the GTF 0768 and vGTFJ enzymes in Samples 2 and 3. The fresh-fermented product samples were sensorially evaluated. Sample 1 was noted to be of the least texture and the sweetest, but also to have a very clear undesirable bitterness and astringent taste. In contrast to Sample 1, Samples 2 and 3 both had increased texture due to the formation of dextran-alpha-1,3-glucan graft copolymer (Sample 2) or linear alpha 1,3-glucan (Sample 3), and were less sweet. Interestingly, both Samples 2 and 3 had a more neutral flavor without the high bitterness and astringent taste observed in Sample 1.

The presence of isoflavones (daidzein, genistein), isoflavone glycosides (daidzin, genistin) and glucosylated isoflavone glycosides (daidzin and genistin modified to have one or more additional glucosyl groups) was assessed for each sample (by LC-MS) according to the Materials/Methods. Herein, an isoflavone glycoside contains a single glucosyl side moiety, whereas a glucosylated isoflavone glycoside has two more glucosyl side moieties; the additional glucosyl group(s) of a glucosylated isoflavone glycoside resulted herein from the activity of added GTF enzyme(s). Isoflavones, isoflavone glycosides, and glucosylated isoflavone glycosides detected in the samples are presented in Table 1. It was found that glucosylated forms of the isoflavone glycosides daidzin (e.g., DaidzinHex2, DaidzinHex3, DaidzinHex4, DaidzinHex5, DaidzinHex6 and DaidzinHex7 presented in Table 1) and genistin (e.g., GenistinHex2, GenistinHex3, GenistinHex4, GenistinHex5 and GenistinHex6 presented in Table 1) were present only in Samples 2 and 3, and contained 2-11 glucosyl groups. Merely as an example to demonstrate the nomenclature of Table 1, “DaidzinHex2” represents daidzin to which one glucosyl has been added, and so this species has a total of two (2) glucosyl groups. It is possible that each detected glucosylated species (DaidzinHex2+, GenistinHex2+) was comprised of glucosylated isoforms (glucosyls added at different sites of the glycoside substrate), or had all glucosyls added linearly at one site of the glycoside substrate (i.e., no isoforms). It was therefore clear that GTF 0768 and vGTFJ were able to glucosylate isoflavone glycosides in situ. This GTF activity was associated with a reduction of bitterness and astringent taste of the fresh-fermented preparation.

TABLE 1

| | Sample 1 | Sample 2 | Sample 3 |
|---|---|---|---|
| Daidzein | 252 | 241 | 244 |
| Daidzin | 12110 | 6927 | 5457 |
| DaidzinHex2 | 0 | 568 | 157 |
| DaidzinHex3 | 0 | 1818 | 403 |
| DaidzinHex4 | 0 | 1310 | 478 |
| DaidzinHex5 | 0 | 785 | 106 |
| DaidzinHex6 | 0 | 480 | 2 |
| DaidzinHex7 | 0 | 100 | 0 |
| Genistein | 14 | 5 | 8 |
| Genistin | 10075 | 6272 | 6052 |
| GenistinHex2 | 0 | 1196 | 350 |
| GenistinHex3 | 0 | 1515 | 422 |
| GenistinHex4 | 0 | 1147 | 506 |
| GenistinHex5 | 0 | 724 | 73 |
| GenistinHex6 | 0 | 276 | 1 |

Note:
The listed values are chromatographic peak areas.

What is claimed is:

1. A method of producing a food product/precursor, said method comprising:
(a) providing a food product/precursor that comprises at least water, sucrose and a plant-based material, and
(b) contacting the food product/precursor with at least:
(i) a glucosyltransferase enzyme that synthesizes alpha-1,6-glucan, wherein at least about 50% of the glycosidic linkages of the alpha-1,6-glucan are alpha-1,6 linkages, and/or
(ii) a glucosyltransferase enzyme that synthesizes alpha-1,3-glucan, wherein at least about 50% of the glycosidic linkages of the alpha-1,3-glucan are alpha-1,3 linkages,
typically wherein at least one alpha-glucan is produced in the food product/precursor,
whereby the food product/food precursor, after step (b), has reduced off-flavor as compared to the food product/precursor before step (b),
and optionally has a reduced sugar content as compared to the food product/precursor before step (b).

2. The method of claim 1, wherein the food product/food precursor, after step (b), has a reduced content of at least one isoflavone glycoside and/or an increased content of at least one glucosylated isoflavone glycoside, as compared to the food product/precursor before step (b),
optionally wherein said reduced off-flavor results from said reduced content of at least one isoflavone glycoside.

3. The method of claim 2, wherein said at least one isoflavone glycoside is daidzin or genistin.

4. The method of claim 1, wherein said off-flavor comprises bitterness and/or astringency.

5. The method of claim 1, wherein:
said glucosyltransferase enzyme that synthesizes alpha-1,6-glucan comprises an amino acid sequence that is at least 90% identical to SEQ ID NO:1, 2, 11, 12, 14, 15, 16, 17, or 18, and/or
said glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO: 7, residues 55-960 of SEQ ID NO:8, residues 55-960 of SEQ ID NO:9, or SEQ ID NO:13.

6. The method of claim 1, wherein step (a) comprises adding sucrose to the food product/precursor.

7. The method of claim 1, wherein the food product/precursor of step (a) is fermented, or
the method further comprises, during or after step (b), fermenting the food product/precursor.

8. The method of claim 1, wherein the food product/precursor is a non-dairy food product/precursor,
optionally wherein the food product/precursor produced by said method is a fresh fermented product/precursor.

9. A food product/precursor produced by the method of claim 1.

10. A method of glucosylating an isoflavone glycoside, said method comprising:
providing a composition that comprises at least water, sucrose, an isoflavone glycoside, and a glucosyltransferase enzyme,
wherein the glucosyltransferase enzyme is selected from:
(i) a glucosyltransferase enzyme that synthesizes alpha-1,6-glucan, wherein at least about 50% of the glycosidic linkages of the alpha-1,6-glucan are alpha-1,6 linkages, and/or
(ii) a glucosyltransferase enzyme that synthesizes alpha-1,3-glucan, wherein at least about 50% of the glycosidic linkages of the alpha-1,3-glucan are alpha-1,3 linkages,
wherein at least one glucosylated form of the isoflavone glycoside is produced in the composition, and typically wherein at least one alpha-glucan is produced in the composition.

11. The method of claim 10, wherein the isoflavone glycoside is daidzin or genistin.

12. The method of claim 10, wherein:
said glucosyltransferase enzyme that synthesizes alpha-1,6-glucan comprises an amino acid sequence that is at least 90% identical to SEQ ID NO:1, 2, 11, 12, 14, 15, 16, 17, or 18, and/or
said glucosyltransferase enzyme that synthesizes alpha-1,3-glucan comprises an amino acid sequence that is at least 90% identical to residues 55-960 of SEQ ID NO:5, residues 54-957 of SEQ ID NO:6, residues 55-960 of SEQ ID NO: 7, residues 55-960 of SEQ ID NO:8, residues 55-960 of SEQ ID NO:9, or SEQ ID NO:13.

13. The method of claim 10, wherein the composition is a food product/precursor.

14. The method of claim 13, wherein the food product/precursor is a plant-based food product/precursor, optionally wherein the food product/precursor is a fresh fermented product/precursor.

15. The method of claim 13, further comprising: fermenting the food product/precursor after said providing step.

16. A composition, or a glucosylated isoflavone glycoside, produced by the method of claim 10.

17. A composition comprising a glucosylated isoflavone glycoside,
wherein the glucosylated isoflavone glycoside is produced by contacting an isoflavone glycoside with a glucosyltransferase enzyme in the presence of at least water and sucrose,
wherein the glucosyltransferase enzyme is selected from:
(i) a glucosyltransferase enzyme that synthesizes alpha-1,6-glucan, wherein at least about 50% of the glycosidic linkages of the alpha-1,6-glucan are alpha-1,6 linkages, and/or
(ii) a glucosyltransferase enzyme that synthesizes alpha-1,3-glucan, wherein at least about 50% of the glycosidic linkages of the alpha-1,3-glucan are alpha-1,3 linkages,
optionally wherein the composition is a food product/precursor.

18. The composition of claim 17, wherein the isoflavone glycoside is daidzin or genistin.
